(19) United States
(12) Patent Application Publication
(10) Pub. No.: US 2023/0274433 A1
(43) Pub. Date: Aug. 31, 2023

(54) MEDICAL INFORMATION PROCESSING SYSTEM, MEDICAL INFORMATION PROCESSING METHOD, AND PROGRAM

(71) Applicant: Sony Group Corporation, Tokyo (JP)

(72) Inventor: Keisuke UYAMA, Tokyo (JP)

(73) Assignee: Sony Group Corporation, Tokyo (JP)

(21) Appl. No.: 18/005,975

(22) PCT Filed: Aug. 18, 2021
(86) PCT No.: PCT/JP2021/030109

§ 371 Date: Jan. 19, 2023

(30) Foreign Application Priority Data
Aug. 31, 2020 (JP) 2020-146085

(51) Int. Cl. G06T 7/00; G06T 1/20; G06V 10/74

(52) U.S. Cl. CPC G06T 7/0014; G06T 1/20; G06V 10/761; G06T 2207/30168

(57) ABSTRACT

The present disclosure relates to a medical information processing system, a medical information processing method, and a program capable of suppressing a decrease in safety due to deterioration in display quality.

When an input image input from a plurality of input devices that supplies various types of medical images is acquired by one signal processing unit, is subjected to predetermined signal processing, and is displayed on any of a plurality of monitor devices by converting to correspond to each display specification, quality of an output image is confirmed from comparison between the input image and the output image subjected to the signal processing, and a warning is presented or signal processing is stopped in a case where deterioration in quality is observed. The present disclosure can be applied to a medical image processing system.

### TECHNICAL FIELD

The present disclosure relates to a medical information processing system, a medical information processing method, and a program, and more particularly, to a medical information processing system, a medical information processing method, and a program capable of suppressing deterioration in safety of medical practice due to deterioration in display quality of a medical image.

### BACKGROUND ART

For the purpose of economic efficiency and information cooperation, various medical image processing devices in a medical environment may be connected to one device such as a server and the like, and each processing result may be displayed on a plurality of monitor devices.

Since display quality of an image displayed by the medical image processing device is related to safety, it is necessary to appropriately manage the display quality.

Therefore, a technique has been proposed in which a band of an image of which display quality is important is prioritized, a compression format is changed, and transfer is performed as a stream (see Patent Document 1).

### CITATION LIST

### Patent Document

Patent Document 1: WO 2020/066972 A SUMMARY OF THE INVENTION

### Problems to be Solved by the Invention

However, while various operations are repeated in a state in which a plurality of image processing devices is connected to one device, there has been a possibility that deterioration in display quality cannot be recognized, safety of medical practice is lowered, and a diagnosis error or a medical accident is induced.

Furthermore, it is possible to set an operation for a plurality of image processing devices after recognizing in advance a degree of allowable deterioration in display quality of a medical image regarding safety. However, in a case where a stream is transferred via a network, the degree of deterioration in the display quality according to a communication state changes. Therefore, it has been difficult to perform appropriate setting according to the communication state.

The present disclosure has been made in view of such a situation, and suppresses a decrease in safety of medical practice due to deterioration in display quality related to medical image display even in a state where a plurality of image processing devices is connected to one device.

### Solutions to Problems

A medical information processing system and a program according to one aspect of the present disclosure are a medical information processing system and a program including: a quality confirmation unit configured to confirm quality of output medical information which is medical information obtained by performing predetermined signal processing on input medical information which is medical information that has been input.

A medical information processing method according to one aspect of the present disclosure is a medical information processing method including: confirming quality of output medical information which is medical information obtained by performing predetermined signal processing on input medical information which is medical information that has been input.

In one aspect of the present disclosure, quality of output medical information which is medical information obtained by performing predetermined signal processing on input medical information which is medical information that has been input is confirmed.

### BRIEF DESCRIPTION OF DRAWINGS

FIG. 1 is a diagram illustrating a configuration example of a medical information processing system.

FIG. 2 is a diagram illustrating a configuration example of a first embodiment of a signal processing unit and a quality confirmation unit.

FIG. 3 is a diagram illustrating a display example of a setting image.

FIG. 4 is a diagram illustrating a display example of a setting image.

FIG. 5 is a flowchart illustrating signal processing by the quality confirmation unit and the signal processing unit in FIG. 2.

FIG. 6 is a diagram illustrating a configuration example of a second embodiment of a quality confirmation unit.

FIG. 7 is a flowchart illustrating signal processing by the quality confirmation unit and a signal processing unit in FIG. 6.

FIG. 8 is a flowchart illustrating a modification of signal processing by the quality confirmation unit and the signal processing unit in FIG. 6.

FIG. 9 is a diagram illustrating a configuration example of a third embodiment of a quality confirmation unit.

FIG. 10 is a diagram illustrating a display example of a setting image.

FIG. 11 is a diagram illustrating a display example of a setting image.

FIG. 12 is a diagram illustrating a display example of a setting image.

FIG. 13 is a diagram illustrating a display example of a setting image.

FIG. 14 is a flowchart illustrating signal processing by the quality confirmation unit and a signal processing unit in FIG. 9.

FIG. 15 is a flowchart illustrating a first modification of signal processing by the quality confirmation unit and the signal processing unit in FIG. 9.

FIG. 16 is a flowchart illustrating a second modification of signal processing by the quality confirmation unit and the signal processing unit in FIG. 9.

FIG. 17 is a diagram illustrating a configuration example of a general-purpose computer.

### MODES FOR CARRYING OUT THE INVENTION

Hereinafter, preferred embodiments of the present disclosure will be described in detail with reference to the accompanying drawings. Note that, in the present specification and the drawings, components having substantially the same functional configuration are designated by the same reference numeral, and duplicate description thereof will be omitted.

Hereinafter, modes for carrying out the present technology will be described. The description will be given in the following order.
1. First Embodiment
2. Second Embodiment
3. Modification of Second Embodiment
4. Third Embodiment
5. First Modification of Third Embodiment
6. Second Modification of Third Embodiment
7. Example Executed by Software

### 1. First Embodiment

<Medical Information Processing System>

The present disclosure suppresses a decrease in safety of medical practice due to deterioration in quality related to processing of a medical image even in a state where a plurality of image processing devices that processes medical images is connected to one device.

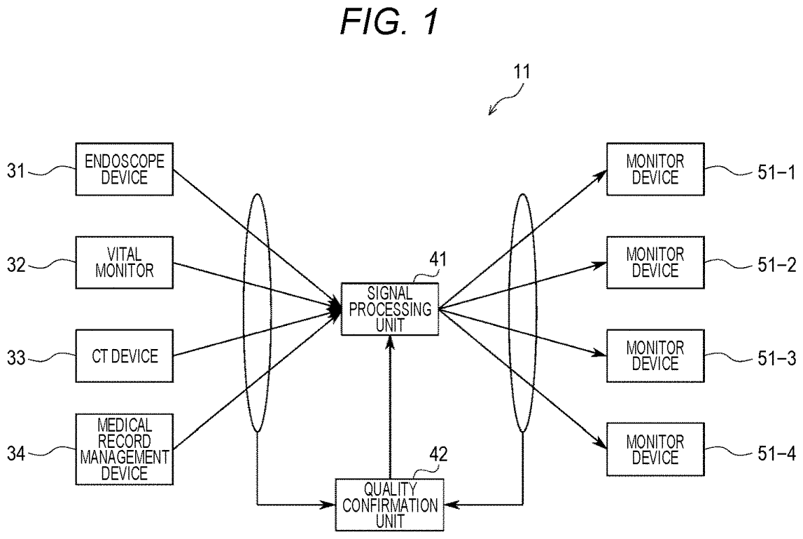

FIG. 1 illustrates a medical information processing system in which medical images are output to a plurality of monitor devices in a state where a plurality of information processing devices that processes the images is connected to one device.

A medical information processing system 11 in FIG. 1 includes an endoscope device 31, a vital monitor 32, a CT device 33, a medical record management device 34, a signal processing unit 41, a quality confirmation unit 42, and monitor devices 51-1 to 51-4.

The endoscope device 31 converts an image captured by a camera provided in an endoscope used for endoscopic surgery into, for example, a low latency video codec (LLVC) and the like, and outputs the image to the signal processing unit 41.

The image supplied from the endoscope device 31 has lower latency than predetermined latency, has higher image quality than predetermined image quality, and has higher importance of a frame rate than predetermined importance.

The vital monitor 32 receives inputs of various vital values such as a body temperature, a blood pressure, and a heart rate, generates an image in a format such as advanced video coding (AVC) and the like, for example, for monitoring, and outputs the image to the signal processing unit 41.

In the image supplied from the vital monitor 32, latency is about predetermined latency, image quality is about predetermined image quality, and importance of a frame rate is higher than predetermined importance.

The computed tomography (CT) device 33 outputs an ultra-high quality image (a high-resolution image) captured by a computed tomography diagnostic apparatus to the signal processing unit 41 at a frame rate lower than a predetermined frame rate while keeping the image uncompressed.

The medical record management device 34 manages medical record information for each patient, and reads the medical record information, thereby outputting the medical record information as an image to the signal processing unit 41.

Since the image output from the medical record management device 34 only needs to be an image that can recognize various types of information, the image is output to the signal processing unit 41 in a compressed state at a frame rate lower than a predetermined frame rate.

The signal processing unit 41 may be configured by, for example, a general computer, or may be configured by a server computer or cloud computing formed on a network.

The signal processing unit 41 performs signal processing set according to an operation input of a user (an operator) input to the quality confirmation unit 42 on an image supplied from at least any one of the endoscope device 31, the vital monitor 32, the CT device 33, and the medical record management device 34. The processed image is output to and displayed on at least any one of the monitor devices 51-1 to 51-4.

More specifically, for example, the signal processing unit 41 performs signal processing of generating an image obtained by pasting an image output from the CT device 33 on an image output from the endoscope device 31 as picture in picture (PnP), and converts the generated image into an image matched with any one designated monitor device 51 of the monitor devices 51-1 to 51-4. The image is output and displayed.

Note that, hereinafter, in a case where it is not necessary to particularly distinguish the monitor devices 51-1 to 51-4, they are simply referred to as a monitor device 51.

The quality confirmation unit 42 receives an operation input from a user (operator) (not illustrated), selects an image supplied from the endoscope device 31, the vital monitor 32, the CT device 33, and the medical record management device 34 connected to the signal processing unit 41 according to the operation input, and determines processing contents for the selected image, thereby setting the processing contents in the signal processing unit 41.

In response to the operation input, the quality confirmation unit 42 confirms quality of an image generated by performing predetermined processing by the signal processing unit 41. The confirmed image is output to and displayed on at least any one of the monitor devices 51-1 to 51-4.

The quality confirmation unit 42 recognizes each of the endoscope device 31, the vital monitor 32, the CT device 33, and the medical record management device 34 that supply images to the signal processing unit 41, and recognizes each of the monitor devices 51-1 to 51-4, thereby confirming quality of an image generated by executing the signal processing according to the operation input on the basis of a processing capability and a processing state of the signal processing unit 41.

In a case where there is a problem in the quality of the confirmed image, the quality confirmation unit 42 presents a warning in the monitor device 51 to which the output of the image is designated.

The quality confirmation unit 42 may stop the signal processing by the signal processing unit 41 according to the quality of the image processed by the confirmed signal processing unit 41.

For example, in a case where an image observed by a surgeon (an operator) performing surgery is displayed on the monitor device 51, when there is a problem in an output image such as that a color of an image generated by the signal processing of the signal processing unit 41 cannot be appropriately represented to such an extent that erroneous diagnosis occurs or that a large delay has occurred in a moving image, the quality confirmation unit 42 presents a warning image indicating that there is a problem in the image generated by the signal processing or indicating that there is a possibility that a problem occurs in the image generated by the signal processing.

The quality confirmation unit 42 may stop the operation itself of the signal processing unit 41 so that the image generated by the signal processing is not output to the monitor devices 51-1 to 51-4, and then display a warning image indicating that a problem occurs in the image generated by the signal processing.

Note that, in the present specification, an example in a case where a processing target of the signal processing unit 41 is a medical image is described. However, the processing target may be other than the image as long as it handles a medical signal, and for example, may be general medical information such as voice and text. Therefore, the processing target of the signal processing unit 41 can also be regarded as medical information including general information handled for medical purposes such as a medical image (including raw data before development and the like), voice, text, and the like.

Therefore, an output destination of the signal processing unit 41 is only required to be an output device that can output a signal processing result of the signal processing unit 41. Therefore, the output destination is not limited to the display device such as the monitor device 51 and the like, and is only required to output various types of medical information such as a speaker, lights, a siren, and a vibrator. The output destination may be a device that executes processing based on the signal processing result of the signal processing unit 41, for example, a medical AI device.

Therefore, the medical information processing system 11 of FIG. 1 can be regarded as suppressing a decrease in safety of medical practice due to deterioration in quality related to processing of medical information even in a state where a plurality of information processing devices that executes processing for general medical information including a medical image, voice, a text, and the like is connected to one device.

Similarly, the endoscope device 31, the vital monitor 32, the CT device 33, and the medical record management device 34 are representative examples of input devices that supply medical images among medical information to the signal processing unit 41, and other configurations may be provided as long as they supply medical information.

In the present specification, a case where information to be processed by the signal processing unit 41 is a medical image will be described, but the present disclosure can be applied to general medical information.

<Configuration Example of Signal Processing of Signal Processing Unit and Quality Confirmation Unit>

(Configuration Example of Quality Confirmation Unit)

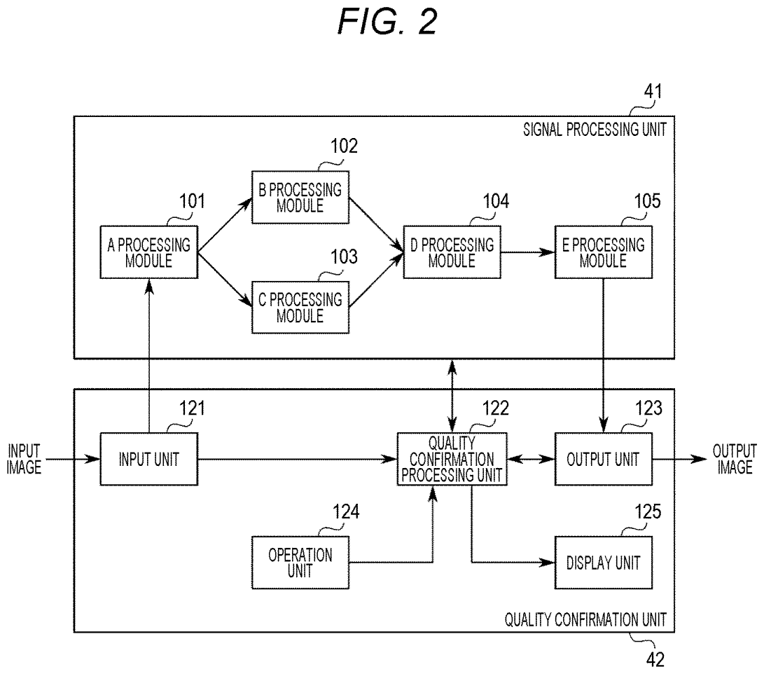

Next, a configuration example of signal processing of the signal processing unit 41 and the quality confirmation unit 42 will be described with reference to FIG. 2.

The signal processing unit 41 is realized by at least any one of a general personal computer, a server computer, or cloud computing, and executes signal processing set by the quality confirmation unit 42 on images supplied from the endoscope device 31, the vital monitor 32, the CT device 33, and the medical record management device 34.

The quality confirmation unit 42 includes an input unit 121, a quality confirmation processing unit 122, an output unit 123, an operation unit 124, and a display unit 125.

The input unit 121 receives an input of an image signal of an image necessary for causing the signal processing unit 41 to execute preset signal processing among images supplied from the endoscope device 31, the vital monitor 32, the CT device 33, and the medical record management device 34, and outputs it to the signal processing unit 41 and the quality confirmation processing unit 122.

Hereinafter, an image input by the input unit 121 and output to the signal processing unit 41 and the quality confirmation processing unit 122 is referred to as an input image.

The output unit 123 receives an input of an image to be a signal processing result of the signal processing unit 41 and outputs the image to the quality confirmation processing unit 122. If an instruction on an output is given from the quality confirmation processing unit 122, the image to be the signal processing result is output to and displayed on at least any one of the monitor devices 51-1 to 51-4 at a subsequent stage.

Hereinafter, an image input from the signal processing unit 41 by the output unit 123 and output to the quality confirmation processing unit 122 and at least any one of the monitor devices 51-1 to 51-4 is referred to as an output image.

The quality confirmation processing unit 122 recognizes what kind of device is connected to the input unit 121 on the basis of any of metadata, data contents, user setting, or the like of the input image supplied from the input unit 121.

In a case of FIG. 1, the quality confirmation processing unit 122 recognizes that each of the endoscope device 31, the vital monitor 32, the CT device 33, and the medical record management device 34 is connected to the input unit 121 on the basis of any information of the metadata, the data contents, and the user setting of the input image input to the input unit 121, and specifies various types of information such as an image size, a frame rate, a codec (compression method), a compression rate, and the like of the supplied image on the basis of a recognition result.

The quality confirmation processing unit 122 can recognize what kind of characteristic a device connected to the output unit 123 has by a signal or the like supplied from the device connected to the output unit 123. For example, in a case where the output unit 123 and the display device are connected by a high-definition multimedia interface (HDMI) (registered trademark) cable, the quality confirmation processing unit 122 recognizes a type of the connected display device and a characteristic thereof on the basis of information included in a synchronization signal defined in a standard of the HDMI (registered trademark) cable to which the display device is connected via the output unit 123.

In the case of FIG. 1, the quality confirmation processing unit 122 recognizes that the devices connected to the output unit 123 are the monitor devices 51-1 to 51-4, and can specify optimum image size, frame rate, codec (compression method), compression rate, and the like according to each characteristic on the basis of a recognition result.

In addition to the image size, the frame rate, the codec (compression method), the compression rate, and the like, the quality confirmation processing unit 122 may specify information of latency, resolution, bit depth, gamma, display luminance, white balance, color gamut, chroma sampling, and an invisible area for the image input from the device connected to the input unit 121 and the image displayed by the device connected to the output unit 123 on the basis of any of the metadata, the data contents, or the user setting of the image supplied from the input unit 121 and the device connected to the output unit 123.

Note that FIG. 1 illustrates a configuration in which an input image is directly supplied from the endoscope device 31, the vital monitor 32, the CT device 33, and the medical record management device 34 to the signal processing unit 41, and an output image is directly output from the signal processing unit 41 to the monitor devices 51-1 to 51-4.

Therefore, according to the configuration of FIG. 1, the input unit 121 may be provided outside the quality confirmation unit 42, and the input unit 121 may output the input image to each of the signal processing unit 41 and the quality confirmation unit 42.

Similarly, according to the configuration of FIG. 1, the output unit 123 may be provided outside the quality confirmation unit 42, and the output unit 123 may output the output image to each of the quality confirmation unit 42 and the monitor devices 51-1 to 5-4.

The quality confirmation processing unit 122 compares the input image supplied from the input unit 121 and the output image supplied from the output unit 123, and detects an image quality change of the output image with respect to the input image, thereby confirming quality of the output image with respect to the input image.

More specifically, the quality confirmation processing unit 122 confirms the quality of the output image by executing, for example, at least any one of comparison processing such as difference pixel comparison, difference statistic comparison, difference comparison by machine learning, and the like as detection of the image quality change between the input image and the output image.

The quality of the output image includes an image quality change of the output image with respect to the input image of at least any one information of latency, resolution, frame rate, bit depth, gamma, display luminance, white balance, color gamut, chroma sampling, a compression method, a compression rate, an invisible area, and the like.

By detecting the image quality change between the input image and the output image, the quality confirmation processing unit 122 confirms the quality of the output image on the basis of, for example, whether or not an increase in latency exceeding a prescribed value to the input image, a decrease or improvement (stretching) in resolution, a change in frame rate, a change in display luminance, image deterioration/image addition, image overlapping, and the like has occurred in the output image.

At this time, the quality confirmation processing unit 122 needs to appropriately select a method of detecting the image quality change between the input image and the output image according to whether a difference that degrades the display quality due to the signal processing or degradation of the image quality caused by the addition of the processing.

That is, in a case where the signal processing by the signal processing unit 41 is processing of generating a PnP image, there is a possibility that important information is hidden behind a small image to be displayed in a superimposed manner, or resolution is deteriorated by setting such that right and left images are displayed side by side and the like in three-dimensional display. In such a case, it can be considered that the display quality is deteriorated due to addition of processing of displaying images in a superimposed manner or processing of displaying images side by side rather than deterioration of the display quality due to the signal processing.

Therefore, in such determination, rather than detection of the image quality itself, a method of analyzing a position of an important subject (for example, an organ or an affected part to be operated on) in the image and verifying whether or not the important subject (for example, an organ or an affected part to be operated on) is not hidden and whether or not resolution has changed by being displayed side by side is required.

An artifact may occur in the signal processing required for the signal processing unit 41 due to, for example, selection of a method associated with reduction or enlargement. As described above, in a case where the method associated with reduction or enlargement is used for the signal processing, the signal processing unit 41 determines what kind of characteristic the input image has by using a discriminator or the like machine-learned in advance, and detects whether or not the input image is an image such as a radiation image, for example, in which a change in display quality should not occur, as an image quality change between the input image and the output image.

In a case where the quality confirmation processing unit 122 determines, on the basis of an image quality change between the input image and the output image, that an image with an image quality that is likely to affect a required medical practice such as an operation and the like of a doctor or a surgeon (operator) performing diagnosis and an image with sufficient quality cannot be obtained, a warning image is displayed, or the signal processing itself by the signal processing unit 41 is stopped to request resetting of processing contents of the signal processing.

The quality confirmation processing unit 122 determines whether or not a type and a format are appropriate as the set input image and output image on the basis of the metadata of each of the input image and the output image, and in a case where the type and the format are not appropriate, a warning image is displayed, or the signal processing itself by the signal processing unit 41 is stopped to request resetting of the processing contents of the signal processing.

For example, in a case where a format of a displayable image is set in the display device connected to the output unit 123, the quality confirmation processing unit 122 determines whether or not the display device connected to the output unit 123 is a displayable format as the output image on the basis of the metadata of the output image. In a case where the format is not appropriate, a warning image is displayed, or the signal processing itself by the signal processing unit 41 is stopped to request resetting of the processing contents of the signal processing.

The quality confirmation processing unit 122 may display a current display condition in an overlay manner on the monitor device 51 as a connection destination at the time of starting the connection, and display a warning image prompting a confirmation operation that there is no problem in a connection condition. When the confirmation operation is not performed, the connection may not be completed.

The quality confirmation processing unit 122 displays a setting image for setting contents of the signal processing executed by the signal processing unit 41 on the display unit 125, and changes the processing contents of the signal processing unit 41 according to an operation signal supplied from the operation unit 124 with respect to the displayed setting image. The signal processing of the signal processing unit 41 is set so that the finally determined signal processing contents are obtained.

The quality confirmation processing unit 122 may determine whether or not the processing contents set as the signal processing of the signal processing unit 41 are appropriate processing contents. In a case where the processing contents are not the appropriate processing contents, a warning image may be displayed, or the signal processing itself by the signal processing unit 41 may be stopped to separately request the setting of the processing contents.

More specifically, for example, the quality confirmation processing unit 122 determines whether or not the set processing contents of the signal processing unit 41 are appropriate processing contents on the basis of whether or not the processing contents conform to a prescribed rule, such as whether or not the processing contents conform to a processing procedure prescribed in the Pharmaceutical Affairs Law.

The operation unit 124 includes a keyboard, a mouse, an operation button, or the like, and is operated by a user (operator) who sets the processing contents of the signal processing by the signal processing unit 41 or executes the signal processing. An operation signal corresponding to the operation contents is generated and output to the quality confirmation processing unit 122.

(Signal Processing of Signal Processing Unit)

The signal processing unit 41 includes a plurality of processing modules that executes signal processing, and includes, for example, an A processing module 101, a B processing module 102, a C processing module 103, a D processing module 104, and an E processing module 105 as illustrated in FIG. 2.

For example, a case will be considered in which the signal processing unit 41 realizes signal processing for generating an image (picture in picture (PnP) image) in which an image output from the CT device 33 is reduced and pasted on an image output from the endoscope device 31 in FIG. 1.

Hereinafter, the image output from the endoscope device 31 is also referred to as an endoscopic image, and the image output from the CT device 33 is also referred to as a CT image.

In a case of FIG. 2, the A processing module 101 converts, for example, the endoscopic image and the CT image into a common image format for image editing, outputs the endoscopic image to the B processing module 102, and outputs the CT image to the C processing module 103.

For example, the B processing module 102 converts the endoscopic image into an image size of the monitor device 51 scheduled to be output at a subsequent stage, and outputs the image to the D processing module 104.

For example, the C processing module 103 converts the CT image into a reduced image size with respect to the image size of the monitor device 51 scheduled to be output at the subsequent stage, and outputs the image to the D processing module 104.

For example, the D processing module 104 superimposes and synthesizes the CT image having the reduced image size at a predetermined position on the endoscopic image having the image size of the monitor device 51, generates a PnP image, and outputs the PnP image to the E processing module 105.

The E processing module 105 converts the generated PnP image into a display format of the monitor device 51 scheduled to be output, and outputs the PnP image as an output image to the output unit 123.

That is, the signal processing of the signal processing unit 41 is executed by a series of signal processing executed by the A processing module 101, the B processing module 102, the C processing module 103, the D processing module 104, and the E processing module 105. The PnP image obtained by superimposing the reduced CT image on a part of the endoscopic image is generated as the output image from the endoscopic image and the CT image which are input images, and is displayed on the designated monitor device 51.

Note that the signal processing unit 41 is merely an example of a configuration set by the quality confirmation unit 42, and for signal processing other than the configuration of FIG. 2, various signal processing can be set by changing order or combination of various processing modules.

<Display Example of Setting Image for Signal Processing of Signal Processing Unit>

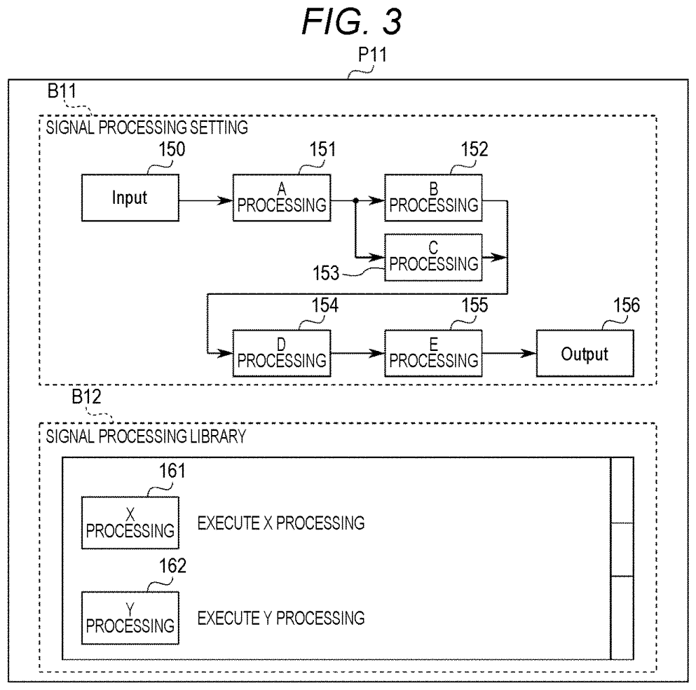

Next, a display example of a setting image for setting signal processing of the signal processing unit 41 will be described with reference to FIG. 3.

In FIG. 3, a setting image P11 is illustrated. The setting image P11 is provided with a signal processing setting field B11 in which “signal processing selection” is written at an upper stage and a signal processing library field B12 in which “signal processing library” is written at a lower stage.

In the signal processing setting field B11, an input block 150 that designates an input image to be a signal processing target indicated as “Input” and an output block 156 that designates an output image to be a signal processing result indicated as “Output” are provided, and signal processing blocks 151 to 155 representing required modularized signal processing are indicated between the input block 150 and the output block 156.

Note that FIG. 3 illustrates a setting example in a case where the signal processing of the signal processing unit 41 in FIG. 2 is realized.

In the signal processing library field B12, signal processing blocks that can be selected as signal processing modules are written together with functional descriptions, and are displayed in a selectable state by operating the operation unit 124.

In the signal processing library field B12 of FIG. 3, an example is illustrated in which a signal processing block 161 denoted as “X processing” and a signal processing block 162 denoted as “Y processing” are displayed.

In the signal processing blocks 161 and 162, “execute X processing” and “execute Y processing” are respectively written as functional descriptions on a right side, and functions of the respective signal processing blocks are described.

In a case of FIG. 3, although not illustrated, information for designating an endoscopic image and a CT image is set in the input block 150 by operating the operation unit 124.

Furthermore, although not illustrated, information for specifying the monitor device 51 whose output is designated and information for designating a PnP image obtained by superimposing a reduced CT image on an endoscopic image are set in the output block 156 by operating the operation unit 124.

In FIG. 3, the signal processing blocks 151 to 155 are selected from the signal processing library field B12 by, for example, an operation such as drag and drop, moved to the signal processing setting field B11, and moreover, connected by arrows so as to designate processing order by operating the operation unit 124, whereby contents of the signal processing of the signal processing unit 41 are set.

Display of the signal processing library field B12 in FIG. 3 can be scrolled, and various selectable signal processing blocks are displayed. Therefore, in the signal processing library field B12 of FIG. 3, the signal processing blocks 151 to 155 are also displayed and can be selected.

More specifically, in the signal processing setting field B11 of FIG. 3, the signal processing block 151 denoted as “A processing” that executes processing of converting into a common image format for image editing is connected after the input block 150, and the signal processing blocks 152 and 153 are connected at a subsequent stage thereof.

Therefore, the processing is set such that the endoscopic image and the CT image specified by the input block 150 are subjected to A processing (processing of converting into a common image format for image editing) corresponding to the A processing module 101 (FIG. 2) in the signal processing block 151, and the endoscopic image converted into the common image format for image editing is output to the signal processing block 152 denoted as “B processing” that executes the processing of the B processing module 102 (FIG. 2).

Furthermore, the processing is set such that the CT image converted into the common image format for image editing is output to the signal processing block 153 denoted as “C processing” that executes the processing of the C processing module 103.

Note that the arrow connected from the signal processing block 151 to the signal processing block 152 is set to include information specifying that the endoscopic image subjected to the format conversion is supplied, and the arrow connected from the signal processing block 151 to the signal processing block 153 is set to include information specifying that the CT image subjected to the format conversion is supplied.

A signal processing block 154 is connected to a subsequent stage of each of the signal processing block 152 denoted as “B processing” and the signal processing block 153 denoted as “C processing”.

Therefore, processing is set such that the endoscopic image undergoes the B processing (processing of converting an endoscopic image into an image size of the monitor device 51 scheduled to be output at a subsequent stage) corresponding to the B processing module 102 (FIG. 2), and the CT image undergoes the C processing (processing of converting the CT image into a reduced image size with respect to the image size of the monitor device 51 scheduled to be output at the subsequent stage) corresponding to the C processing module 103 (FIG. 2). The images are output to the signal processing block 154 denoted as “D processing”.

The signal processing block 154 corresponding to the D processing module 104 (FIG. 2) denoted as “D processing”, which generates a PnP image by superimposing the CT image having the reduced image size at a predetermined position on the endoscopic image having the image size of the monitor device 51, is connected to subsequent stages of the signal processing blocks 152 and 153, and the signal processing block 155 is connected to a subsequent stage of the signal processing block 154.

Therefore, processing is set such that the D processing (processing of superimposing a CT image of a reduced image size at a predetermined position on an endoscopic image of an image size of the monitor device 51 to generate a PnP image) corresponding to the D processing module 104 (FIG. 2) is performed, and the generated PnP image is output to the signal processing block 155 denoted as “E processing”.

The signal processing block 155 corresponding to the E processing module 105 (FIG. 2) denoted as “E processing”, which converts the generated PnP image into a display format of the monitor device 51 scheduled to be output, is connected to the subsequent stage of the signal processing block 154, and is connected to the output block 156 indicating that this processing result is output as an output image.

Therefore, the processing is set so that the E processing (processing of converting a PnP image into a display format of the monitor device 51 scheduled to be output) corresponding to the E processing module 105 (FIG. 2) is performed, and the PnP image as the output image is output from the output block 156 as a signal processing result.

Note that the setting example of the signal processing indicated in the signal processing setting field B11 is merely an example, and the signal processing realized by the signal processing unit 41 can be changed to various forms by selecting any of various signal processing modules prepared in advance in the signal processing library field B12, and furthermore, changing the connection of the selected signal processing modules.

<Display Example of Warning Image>

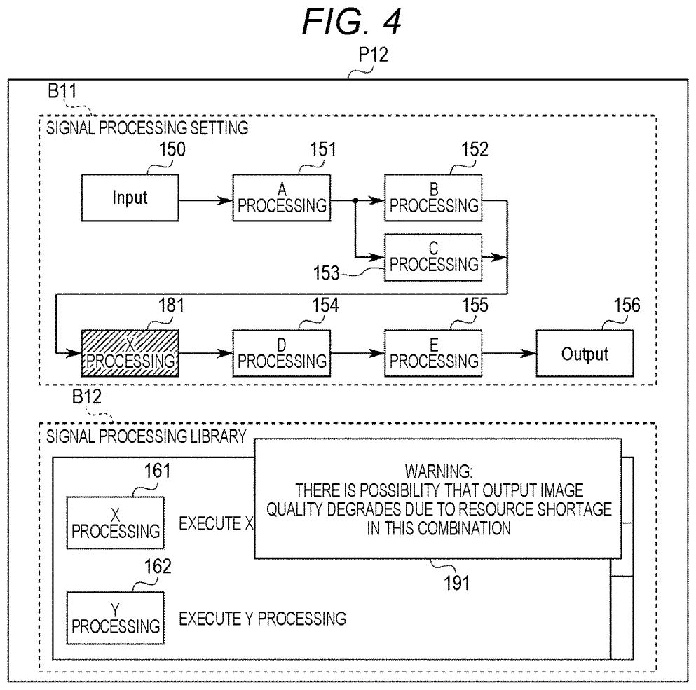

Next, a display example of a warning image will be described with reference to FIG. 4.

The quality confirmation processing unit 122 displays a warning image as illustrated in FIG. 4, for example, in a case where it is determined that image quality is likely to affect required medical practice such as an operation and the like of a doctor or a surgeon (operator) who performs diagnosis, that is, sufficient image quality cannot be obtained, on the basis of an image quality change between an input image and an output image.

FIG. 4 illustrates an example in which a warning field 191 indicated as “there is a possibility that output image quality degrades in this combination” is provided on the signal processing library field B12 in a setting image P12 (corresponding to the setting image P11 in FIG. 3).

Note that, although FIG. 4 illustrates an example in which the warning image is displayed on the display unit 125 of the quality confirmation unit 42, the warning image in which the warning field 191 is displayed may be displayed on the monitor device 51 to which an instruction to output an image is given.

<Signal Processing by Quality Confirmation Unit in FIG. 2>

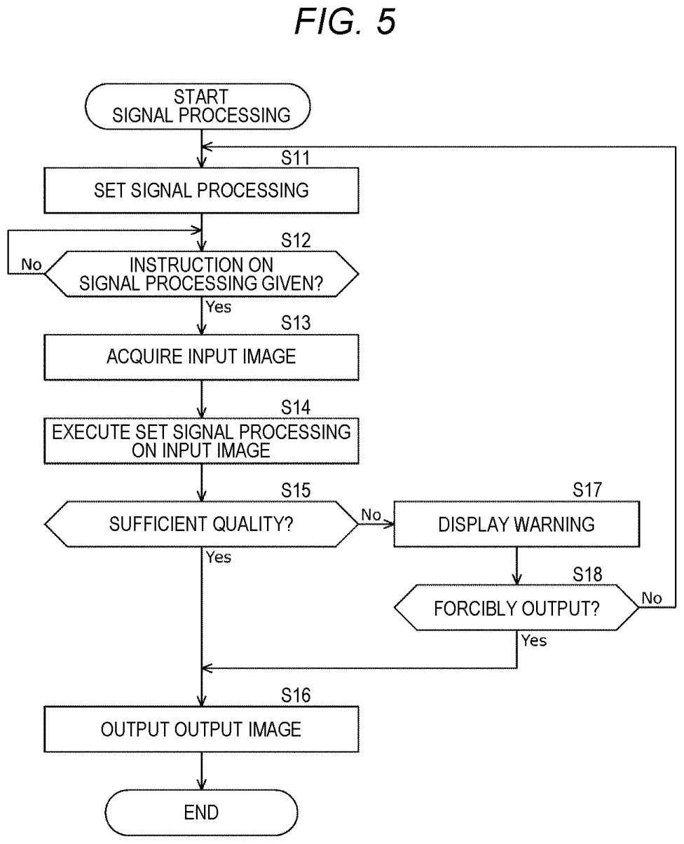

Next, signal processing of the first embodiment by the quality confirmation unit 42 of FIG. 2 will be described with reference to a flowchart of FIG. 5.

In step S11, the quality confirmation processing unit 122 displays a setting image as illustrated by the setting image P11 in FIG. 3 on the display unit 125, and receives an operation input of the operation unit 124, thereby setting processing contents of the signal processing of the signal processing unit 41.

In step S12, the quality confirmation processing unit 122 determines whether or not the operation unit 124 has been operated and an instruction to cause the signal processing unit 41 to execute the set signal processing has been made, and repeats similar processing until the instruction to execute is made. Then, if the instruction on the signal processing by the signal processing unit 41 is given in step S12, the processing proceeds to step S13.

In step S13, the quality confirmation processing unit 122 controls the input unit 121 to receive and acquire a designated input image.

In step S14, the quality confirmation processing unit 122 controls the input unit 121 to supply the acquired input image to the signal processing unit 41 to execute the set signal processing, and to supply the input image to itself. At this time, the signal processing unit 41 performs the set signal processing on the input image to output an output image as a signal processing result to the output unit 123.

In step S15, the quality confirmation processing unit 122 acquires, for example, the output image that is the signal processing result of the signal processing unit 41 from the output unit 123, scores a difference between the input image and the output image, and determines whether or not an image quality change of the output image with respect to the input image is sufficiently small and the output image is an image of sufficient quality that satisfies criteria required for realizing medical practice from comparison between the obtained score and a predetermined threshold (first threshold).

Note that, here, in scoring, for example, the difference between the input image and the output image may be a sum of differences in pixel values between corresponding pixels, for example, as a score.

For the scoring, a degree of increase in latency, a degree of reduction or improvement in resolution, and a degree of change in frame rate between the input image and the output image may be scored.

For example, as to whether or not a format of the output image is according to a requested format, a predetermined score such as a score of 0 in a case of matching and a score of 100 in a case of not matching may be assigned.

As to whether or not the signal processing contents of the signal processing unit 41 conform to a prescribed rule, a predetermined score such as a score of 0 in a case of conforming to the prescribed rule and a score of 50 in a case of not conforming to the prescribed rule may be assigned.

Regarding whether or not the signal processing contents of the signal processing unit 41 are in a state in which a subject that needs to be viewed is overlapped and invisible in signal processing for generating a PnP image, a score may be set to 0 in a case where the subject that needs to be viewed is not completely hidden, and a score according to an area ratio of a hidden portion may be set in a case where only a part of the subject that needs to be viewed is hidden.

Furthermore, the score indicating the difference between the input image and the output image may be used in combination by normalizing the scores of the plurality of elements described above.

In step S15, if the score corresponding to the difference between the input image and the output image is smaller than the predetermined threshold (first threshold) and it is determined that the output image is an image of sufficient quality, the processing proceeds to step S16.

In step S16, the quality confirmation processing unit 122 controls the output unit 123 to output and display the output image on the designated monitor device 51.

On the other hand, in step S15, if the score corresponding to the difference between the input image and the output image is not smaller than the predetermined threshold (first threshold) and it is determined the output image is not an image of sufficient quality, the processing proceeds to step S17.

In step S17, the quality confirmation processing unit 122 controls, for example, the display unit 125 to display a warning image indicating that the quality of the output image is not sufficient, as described with reference to FIG. 4.

In step S18, the quality confirmation processing unit 122 determines whether or not the output image is forcibly output and displayed though the quality thereof is not sufficient.

At this time, the quality confirmation processing unit 122 may determine whether or not to forcibly output the image on the basis of, for example, comparison between a score corresponding to the image quality change of the output image with respect to the input image and a predetermined threshold larger than the predetermined threshold (second threshold (> first threshold)) in step S15.

That is, the quality confirmation processing unit 122 determines whether or not the image is in a range in which there is no problem even if the image quality change is presented to a viewer on the assumption that the score corresponding to the image quality change of the output image with respect to the input image is larger than the predetermined threshold (first threshold) in step S15 but smaller than the predetermined threshold smaller than the predetermined threshold (second threshold (> first threshold)), and the output image does not satisfy the criteria required in realizing the medical practice, but the warning about the image quality change with respect to the input image is displayed.

In a case where it is determined in step S18 that the quality of the output image is not sufficient but the output image is forcibly output, the processing proceeds to step S16. The output image is displayed on the designated monitor device 51 in a state where the warning image presented in the processing of step S17 is displayed on the display unit 125.

For example, in a case where the score corresponding to the image quality change and the threshold are compared in step S18, if the score corresponding to the image quality change of the output image with respect to the input image is larger than the predetermined threshold (first threshold) in step S15 but smaller than the predetermined threshold smaller than the predetermined threshold (second threshold (> first threshold)), and it is determined that the image may be forcibly output, that is, if the output image does not satisfy the criteria required for realizing the medical practice but is regarded as an image in a range in which there is no problem even if the image quality change is presented to the viewer on the assumption that the warning about the image quality change with respect to the input image is displayed, the processing proceeds to step S16.

Furthermore, in step S18, in a case where the quality of the output image is not sufficient and thus it is determined that the output image is not forcibly output and displayed, that is, for example, in a case where the score corresponding to the image quality change of the output image with respect to the input image is larger than the second threshold (> the first threshold) and it is determined that the image should not be forcibly output, the processing returns to step S11, and the subsequent processing is repeated.

That is, in this case, a change of setting of the contents of the signal processing of the signal processing unit 41 and the signal processing by the signal processing unit 41 are repeated until it is considered that the output image satisfies sufficient quality or may be displayed on the premise that it is displayed together with the warning image.

By the above processing, it is possible to freely set desired signal processing, and it is possible to present an output image of sufficient quality by determining whether or not the image quality change of the output image with respect to the input image is large by the set signal processing.

Furthermore, the warning image is presented when the output image generated by performing the signal processing on the input image is not an image of sufficient quality, so that the viewer can view the image after recognizing that the displayed image is not an output image of sufficient quality.

Moreover, in a case of an inappropriate output image, such as a case of an image with a level of quality that cannot be recognized even if displayed together with a warning image, a case where an image has not been converted into an appropriate format, a case of signal processing that has not conformed to a prescribed rule, and the like, resetting of the contents of the signal processing of the signal processing unit 41 is prompted. Therefore, the contents of the signal processing can be reset so that an output image with sufficient quality is output.

For example, in a case where a PnP image is generated by reducing and superimposing a CT image on an endoscopic image, a delay is increased by reducing and superimposing the CT image. If a delay also occurs in the endoscopic image, the image appears to be normally displayed unless there is a warning with respect to quality of an output image. Therefore, there has been a possibility that an abnormality is not noticed and a medical accident occurs.

However, by the series of processing described above, the quality of the output image is confirmed, and a warning is issued that the quality is not sufficient. Therefore, it is possible to cause a user (medical personnel (for example, an operator)) to recognize a dangerous state that adversely affects medical practice. As a result, it is possible to prevent a medical accident.

Furthermore, since the quality of the output image is confirmed on the basis of the difference between the input image and the output image, it is possible to determine whether or not the output image has sufficient quality even in signal processing including a combination of complicated processing that is difficult for a designer to grasp.

Moreover, restriction of clean areas, unclean areas, and the like, and a role in surgery are determined in a medical site, and thus, an operator (a technician or the like) of the signal processing unit 41 and a viewer (an operator or the like) of an image displayed on the monitor device 51 may be different from each other.

For this reason, the viewer cannot grasp what kind of signal processing has generated the image displayed on the monitor device 51, and it is often difficult to recognize what kind of problem has occurred in the output image.

However, according to the present disclosure, the quality of the output image is confirmed, and resetting of the signal processing contents of the signal processing unit 41 can be urged as necessary, or the output image can be displayed by presenting a warning that there is a problem in the quality of the output image. Therefore, it is possible to prevent a human error caused by a fact that the problem occurring in the output image cannot be recognized.

Note that, in the processing of step S18, an example in which the determination is made based on the score has been described. However, an image inquiring of a user whether or not to forcibly output and display the output image may be displayed on the display unit 125, and the processing may be performed according to an operation input of the corresponding user. Furthermore, whether or not to forcibly output the output image may be set in advance as the processing contents of the signal processing.

### 2. Second Embodiment

In the above description, an example has been described in which whether or not an output image is an image of sufficient quality is determined on the basis of an image quality change of the output image with respect to an actual input image.

However, before the signal processing based on the actual input images from the endoscope device 31, the vital monitor 32, the CT device 33, and the medical record management device 34 is executed, signal processing by the signal processing unit 41 may be executed using a reference image that is an image specified in advance as an input image to obtain an image quality change of an output image that is a signal processing result with respect to the reference image (input image), and whether or not an output image is an image of sufficient quality may be determined on the basis of the image quality change.

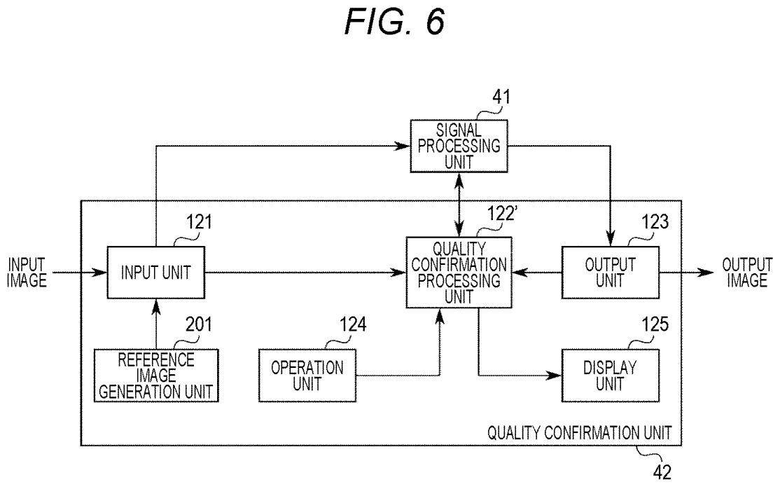

FIG. 6 is a configuration example of the quality confirmation unit 42 using the reference image as the input image.

Note that, in the quality confirmation unit 42 in FIG. 6, a configuration having the same function as the configuration of the quality confirmation unit 42 in FIG. 2 is denoted by the same reference numeral, and description thereof will be appropriately omitted.

That is, the quality confirmation unit 42 in FIG. 6 is different from the quality confirmation unit 42 in FIG. 2 in that a quality confirmation processing unit 122′ is provided instead of the quality confirmation processing unit 122, and moreover, a reference image generation unit 201 is newly provided.

The reference image generation unit 201 generates a resolution chart, a color sample chart, and the like as a reference image, and outputs the reference image to the input unit 121.

That is, since the resolution chart or the like is used as the reference image, for example, information such as how many thin lines are present in a specific region of the resolution chart is clear in advance. Therefore, in the output image, quality of the output image with respect to the resolution can be confirmed in detail by comparison with the number of thin lines recognized in the same region.

Furthermore, since the color sample chart or the like is used as the reference image, for example, information such as a color of a specific region of the color sample chart is clear in advance. Therefore, in the output image, it is possible to confirm quality of the output image with respect to the color in detail by comparison with the color recognized in the same region.

The quality confirmation processing unit 122′ basically has the same function as the quality confirmation processing unit 122, but in confirming the quality of the output image, the input unit 121 is controlled to acquire a reference image from the reference image generation unit 201 instead of the input image.

The quality confirmation processing unit 122′ determines whether or not the output image is an image of sufficient quality on the basis of a difference between the reference image corresponding to the input image and the output image obtained by performing the signal processing on the reference image by the signal processing unit 41.

On the basis of the difference between the reference image corresponding to the input image and the output image obtained by performing the signal processing on the reference image by the signal processing unit 41, when the output image is an image of sufficient quality, the quality confirmation processing unit 122′ controls the input unit 121 to acquire an actual input image set as processing contents of the signal processing, causes the signal processing unit 41 to execute the signal processing, and controls the output unit 123 to output an output image as a signal processing result to the designated monitor device 51 to be displayed.

<Signal Processing by Quality Confirmation Unit in FIG. 6>

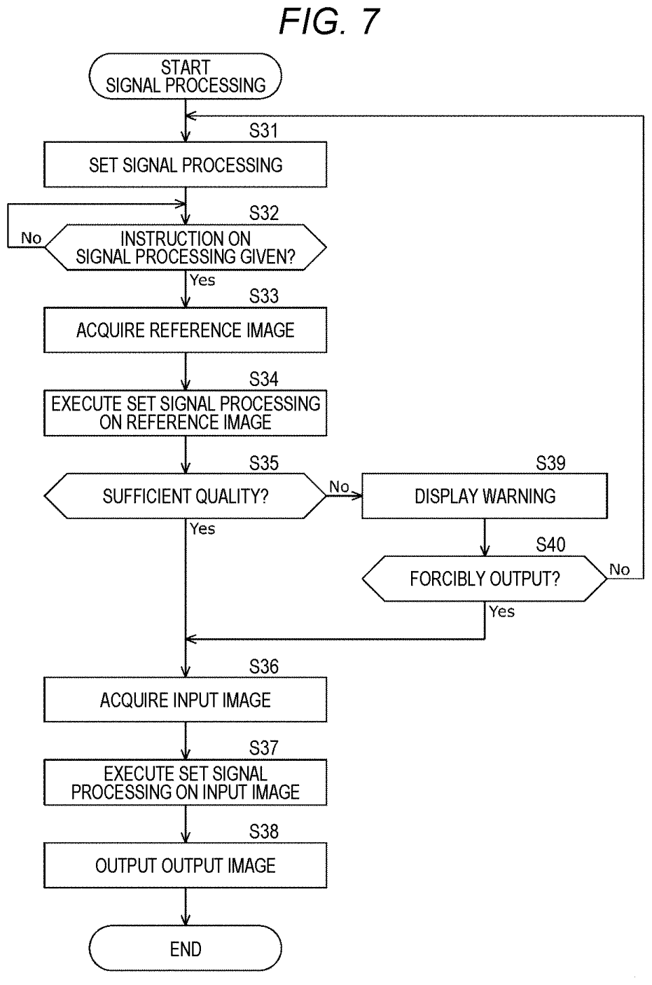

Next, signal processing of the second embodiment by the quality confirmation unit 42 of FIG. 6 will be described with reference to a flowchart of FIG. 7.

In step S31, the quality confirmation processing unit 122′ displays a setting image as illustrated by the setting image P11 in FIG. 3 on the display unit 125, and receives an operation input of the operation unit 124, thereby setting processing contents of the signal processing by the signal processing unit 41.

In step S32, the quality confirmation processing unit 122′ determines whether or not the operation unit 124 has been operated and an instruction to cause the signal processing unit 41 to execute the set signal processing has been made, and repeats similar processing until the instruction to execute is made. Then, if the instruction on the signal processing by the signal processing unit 41 is given in step S32, the processing proceeds to step S33.

In step S33, the quality confirmation processing unit 122′ controls the input unit 121 to acquire a reference image generated by the reference image generation unit 201.

In step S34, the quality confirmation processing unit 122′ controls the input unit 121 to supply the acquired reference image to the signal processing unit 41 to execute the set signal processing, and to supply the reference image to itself. At this time, the signal processing unit 41 performs the set signal processing on the reference image to output an output image for the reference image as a signal processing result to the output unit 123.

In step S35, the quality confirmation processing unit 122′ acquires the output image for the reference image that is the signal processing result of the signal processing unit 41 from the output unit 123, scores a difference between the reference image and the output image for the reference image, and determines whether or not an image quality change of the output image with respect to the input image is sufficiently small and the output image is an image of sufficient quality that satisfies criteria required for realizing medical practice from comparison between the obtained score and a predetermined threshold (first threshold).

If it is determined in step S35 that the image quality change of the output image with respect to the input image is sufficiently small and the output image is an image of sufficient quality, the processing proceeds to step S36.

In step S36, the quality confirmation processing unit 122′ controls the input unit 121 to receive and acquire an input image from a designated device (in a case of FIG. 1, at least any one of the endoscope device 31 to the medical record management device 34).

In step S37, the quality confirmation processing unit 122′ controls the input unit 121 to supply the acquired input image to the signal processing unit 41 to execute the set signal processing, and to supply the input image to itself. At this time, the signal processing unit 41 performs the set signal processing on the input image to output an output image as a signal processing result to the output unit 123.

In step S38, the quality confirmation processing unit 122′ controls the output unit 123 to output and display the output image on the designated monitor device 51.

On the other hand, if it is determined in step S35 that the output image is not an image of sufficient quality, the processing proceeds to step S39.

In step S39, the quality confirmation processing unit 122′ controls the display unit 125 to display a warning image indicating that the output image is not an image of sufficient quality, for example, as described with reference to FIG. 4.

In step S40, the quality confirmation processing unit 122′ determines whether or not the output image is forcibly output and displayed though the quality thereof is not sufficient.

In step S40, in a case where it is determined that the output image is forcibly output, that is, in a case where it is considered that the output image does not satisfy the criteria required for realizing the medical practice but is regarded as an image in a range in which there is no problem even if the image quality change is presented to a viewer on the premise that the warning about the image quality change with respect to the input image is displayed, the processing proceeds to step S36. The output image is displayed on the designated monitor device 51 in a state where the warning image presented in the processing of step S39 is displayed.

Furthermore, in a case where it is determined in step S40 that the output image is not forcibly output, the processing returns to step S31, and the subsequent processing is repeated. That is, in this case, a change of setting of the contents of the signal processing and the signal processing in the signal processing unit 41 are repeated until it is considered that the output image satisfies sufficient quality or may be displayed on the premise that it is displayed together with the warning image.

By the above processing, it is possible to freely set desired signal processing, and it is possible to present an output image of sufficient quality by determining whether or not the image quality change of the output image with respect to the reference image is large by the set signal processing.

At this time, since it is possible to confirm whether or not an image having sufficient quality is output as an output image by the set signal processing on the basis of the reference image in advance, it is possible to confirm quality of the output image with higher accuracy.

### 3. Modification of Second Embodiment

<Modification of Signal Processing by Quality Confirmation Unit in FIG. 6>

In the above, an example has been described in which whether or not an output image is an image of sufficient quality is determined on the basis of an image quality change obtained from a difference between a reference image and the output image with respect to the reference image. However, processing of determining whether or not a fatal problem exists in the output image may be added to a preceding stage of determining whether or not the output image is an image of sufficient quality on the basis of the image quality change, and resetting of the signal processing may be immediately urged in a case where the fatal problem exists.

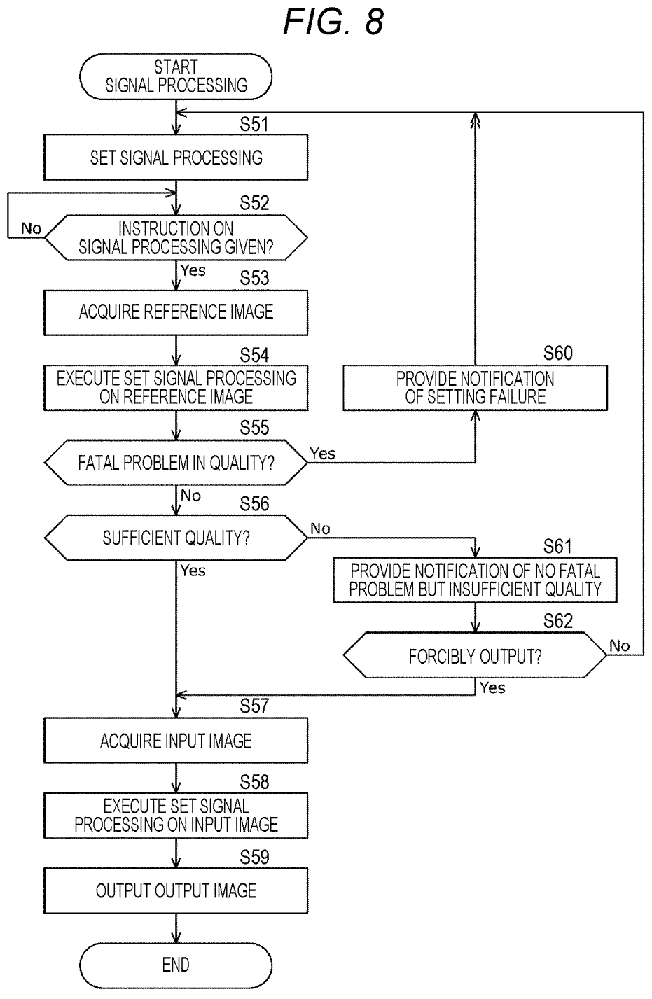

Therefore, next, with reference to a flowchart of FIG. 8, signal processing will be described in which processing of determining whether or not a fatal problem exists in an output image is added to a preceding stage of determining whether or not the image has sufficient quality on the basis of image quality change.

Note that, among the processing in the flowchart of FIG. 8, description of the same processing as the processing in the flowchart of FIG. 7 is appropriately omitted, and the same applies to description of subsequent flowcharts.

If a score related to an image quality change is obtained from a difference between a reference image and an output image with respect to the reference image by processing of steps S51 to S54, the processing proceeds to step S55.

In step S55, the quality confirmation processing unit 122′ acquires an output image that is a signal processing result of the signal processing unit 41 from the output unit 123, and determines whether or not a fatal problem has not occurred in the output image.

The image having the fatal problem is, for example, an entirely black image in which all pixels are equal to or less than a predetermined threshold, an entirely white image in which all pixels are higher than a predetermined threshold, or the like. Furthermore, the image having the fatal problem is, for example, an image in which an increase in latency is larger than a predetermined value, an image in which a decrease or improvement (stretching) in resolution is larger than a predetermined degree, an image in which a change in frame rate, a change in display luminance, and image deterioration are larger than predetermined degrees, an image in which a subject that needs to be displayed is completely hidden due to image addition, image overlapping, or the like, an image in which a predetermined contrast cannot be obtained, an image that is not in a predetermined format, an image obtained by signal processing that is not by a processing procedure specified in the Pharmaceutical Affairs Law, or the like.

In a case where it is considered in step S55 that there is a fatal problem in the output image, the processing proceeds to step S60.

In step S60, the quality confirmation processing unit 122′ controls the display unit 125 to display an image providing notification that processing contents of the signal processing set in the signal processing unit 41 cannot be set.

Then, in a case where it is considered in step S55 that there is no fatal problem, the processing proceeds to processing in and after step S56 where it is determined whether or not the image has sufficient quality and processing according to a determination result is performed.

Here, in a case where it is considered in step S56 that the quality of the image is not sufficient, the processing proceeds to step S61.

In step S61, the quality confirmation processing unit 122′ controls the display unit 125 to display a warning image providing notification that there is no fatal problem but the image is not of sufficient quality.

By the above processing, in a case where the fatal problem has occurred before it is confirmed whether or not the quality of the output image is sufficient, it is possible to warn that the processing contents of the signal processing set in the signal processing unit 41 cannot be set. Therefore, it is not necessary to determine whether or not the image has sufficient quality, it is possible to immediately urge resetting of the signal processing that cannot be permitted regarding safety, and it is possible to smoothly implement trial and error related to the processing contents of the signal processing in the signal processing unit 41.

### 4. Third Embodiment

In the above description, an example has been described in which whether or not an output image obtained by signal processing is an image of sufficient quality is determined from a difference between a reference image and the output image generated by performing signal processing on the reference image. Moreover, the quality of the output image may be confirmed in consideration of resources related to the signal processing in the signal processing unit 41.

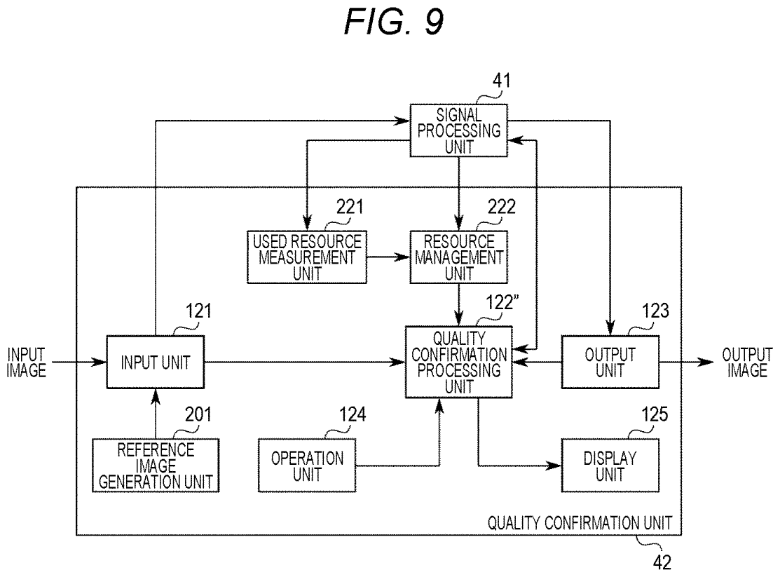

FIG. 9 illustrates a configuration example of the quality confirmation unit 42 configured to confirm quality of an output image in consideration of resources related to signal processing in the signal processing unit 41.

Note that, in the quality confirmation unit 42 in FIG. 9, a configuration having the same function as the configuration of the quality confirmation unit 42 in FIG. 6 is denoted by the same reference numeral, and description thereof will be appropriately omitted.

That is, the quality confirmation unit 42 in FIG. 9 is different from the quality confirmation unit 42 in FIG. 6 in that a quality confirmation processing unit 122″ is provided instead of the quality confirmation processing unit 122′, and a used resource measurement unit 221 and a resource management unit 222 are newly provided.

The used resource measurement unit 221 measures current usage status of various resources including operation resources such as a central processing unit (CPU), a graphics processing unit (GPU), a digital signal processor (DSP), and an encoder/decoder constituting the signal processing unit 41, a memory capacity/band resource, an internal transmission bus band resource, a communication band resource, and the like. A measurement result is supplied to the resource management unit 222.

The resource management unit 222 reads information of a reference resource among various resources including the operation resources such as the central processing unit (CPU), the graphics processing unit (GPU), the digital signal processor (DSP), and the encoder/decoder constituting the signal processing unit 41, the memory capacity/band resource, the internal transmission bus band resource, the communication band resource, and the like. The information is output to the quality confirmation processing unit 122″.

The resource management unit 222 calculates a currently available remaining resource capacity on the basis of the read information of the reference resource and information on the usage status of the resource of the signal processing unit 41 supplied from the used resource measurement unit 221, and outputs the calculated remaining resource capacity to the quality confirmation processing unit 122″.

The quality confirmation processing unit 122″ basically has a function similar to the function of the quality confirmation processing unit 122′. Moreover, the quality confirmation processing unit 122″ presents information on selected signal processing and the resource corresponding to each selected signal processing when displaying a setting image of signal processing of the signal processing unit 41.

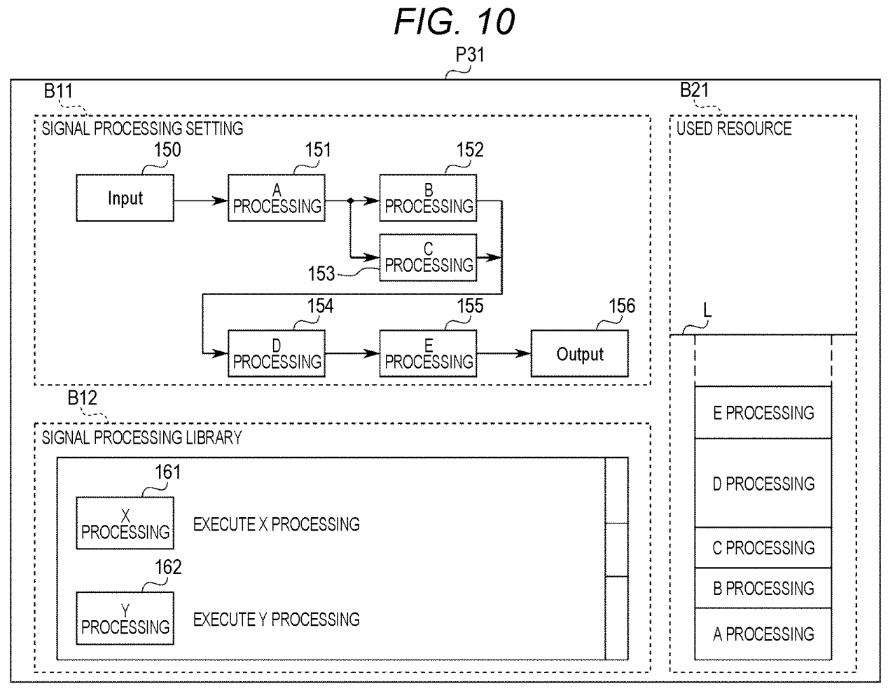

That is, the quality confirmation processing unit 122″ displays a setting image P21 as illustrated in FIG. 10, for example.

Note that, in the setting image P21 in FIG. 10, a configuration having the same function as that of the setting image P11 in FIG. 3 is denoted by the same reference numeral, and description thereof will be appropriately omitted.

That is, the setting image P21 in FIG. 10 is different from the setting image P11 in FIG. 3 in that a used resource field B21 is newly provided.

In the used resource field B21, an occupancy rate of each resource of a currently selected processing module is expressed by an area size from the bottom in descending order of high importance.

That is, in the used resource field B21 in the setting image P11 in FIG. 10, columns corresponding to the signal processing blocks 151 to 155, which are denoted as “A processing”, “B processing”, “C processing”, “D processing”, and “E processing” from the bottom, are denoted in sizes according to the occupancy rates of the resources.

Therefore, in the used resource field B21 of FIG. 10, it is indicated that the importance of the signal processing is high in the order of the A processing, the B processing, the C processing, the D processing, and the E processing, the resources of the B processing and the C processing are the smallest, and next, the resources of the A processing and the E processing are the second largest, and the resource of the D processing is the largest.

Furthermore, a position where the resource is 100% is expressed as a boundary L. Therefore, in FIG. 10, it is indicated that an area of a blank column surrounded by a dotted line from a range described as the E processing to the boundary L is a remaining resource.

Note that, in the setting image P21 of FIG. 10, an example in which the occupancy rate of the resource of each processing module is expressed by a bar graph is illustrated. However, the occupancy rate of the resource for each processing module, the total resource, and the remaining resource may be expressed by a graph other than the bar graph, for example, may be expressed by a circular graph or the like.

If any one of the signal processing blocks is selected from the signal processing library field B12 at the time of setting the signal processing of the signal processing unit 41, the quality confirmation processing unit 122″ obtains the order of importance among signal processing blocks including a currently selected processing block, and writes a column having an area corresponding to an occupancy of a resource of the selected signal processing.

The quality confirmation processing unit 122″ estimates whether or not the signal processing by the signal processing unit 41 requested to be set can be executed on the basis of information of the remaining resource.

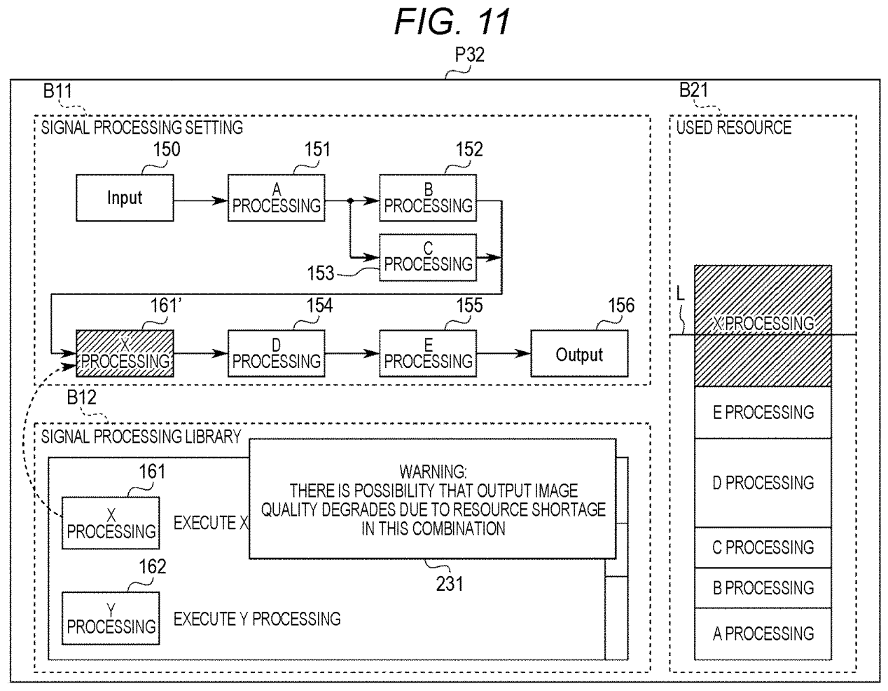

When estimating that there is a possibility that the set signal processing by the signal processing unit 41 cannot be executed on the basis of the information of the remaining resource, the quality confirmation processing unit 122″ controls the display unit 125 to present an image for warning that there is a possibility that a resource is insufficient and there is a possibility that the set signal processing cannot be realized, for example, as illustrated by a setting image P32 in FIG. 11.

In the setting image P32 of FIG. 11, an example is illustrated in which the signal processing block 161 indicated as “X processing” in the signal processing library field B12 is selected, and a signal processing block 161′ is set between the signal processing block 152 indicated as “B processing”, the signal processing block 153 indicated as “C processing” and the signal processing block 154 indicated as “D processing” in the signal processing setting field B11.

The quality confirmation processing unit 122″ calculates a priority and a resource of the newly set signal processing block 161′ indicated as “X processing”, and displays them as a column indicated as “X processing” in the used resource field B21.

In the used resource field B21 in FIG. 11, it is indicated that the signal processing block 161′ indicated as “X processing” has the lowest priority but has a high resource occupancy rate, the signal processing of the signal processing unit 41 as a whole exceeds the boundary L, and the resource exceeds 100% as a whole.

As illustrated in FIG. 11, in a case where there is a possibility that the resource exceeds 100% as a whole, the quality confirmation processing unit 122″ displays a warning field 231 for warning a possibility of degradation of output image quality due to resource shortage on the signal processing library field B12 by a notation “Warning: There is a possibility that output image quality degrades due to resource shortage in this combination”.

With such a warning image, a user can recognize that there is a possibility that the resource shortage occurs and the output image quality deteriorates if the signal processing block 161 indicated as “X processing” is selected as the signal processing.

Note that, in FIG. 11, the signal processing block 161′ and the column denoted as “X processing” are displayed by shaded portions, so that currently selected signal processing is highlighted.

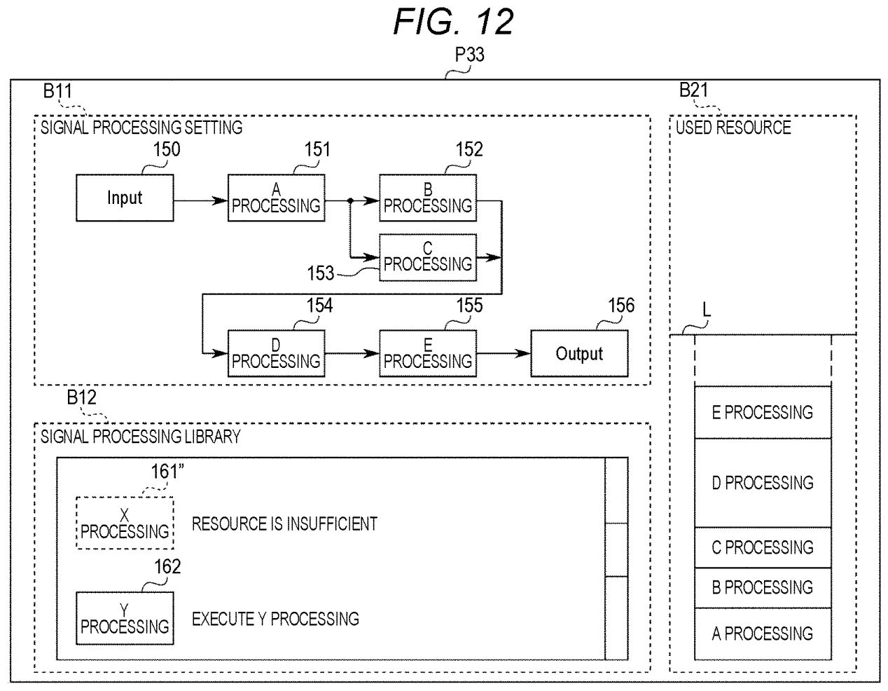

At the time of setting the signal processing, for signal processing of which resource is expected to be insufficient if selected from the information of the remaining resource, the quality confirmation processing unit 122″ may present that the signal processing cannot be selected because there is a possibility that the resource will be insufficient, as illustrated by a setting image P33 of FIG. 12, or may perform display such that the corresponding signal processing cannot be selected.

That is, in the signal processing library field B12 of the setting image P33 in FIG. 12, a signal processing block 161″ indicated as “X processing” is displayed by a dotted line, and it is indicated that resource shortage occurs and selection cannot be performed if this signal processing is selected.

In a case where set signal processing is executed, the quality confirmation processing unit 122″ grasps usage status of a resource related to the signal processing and controls the display unit 125 to display a warning image when recognizing that the usage status of the resource becomes 100% and there is no remaining resource, or when there is a possibility that an output image is not an image of sufficient quality from a reference image and the output image.

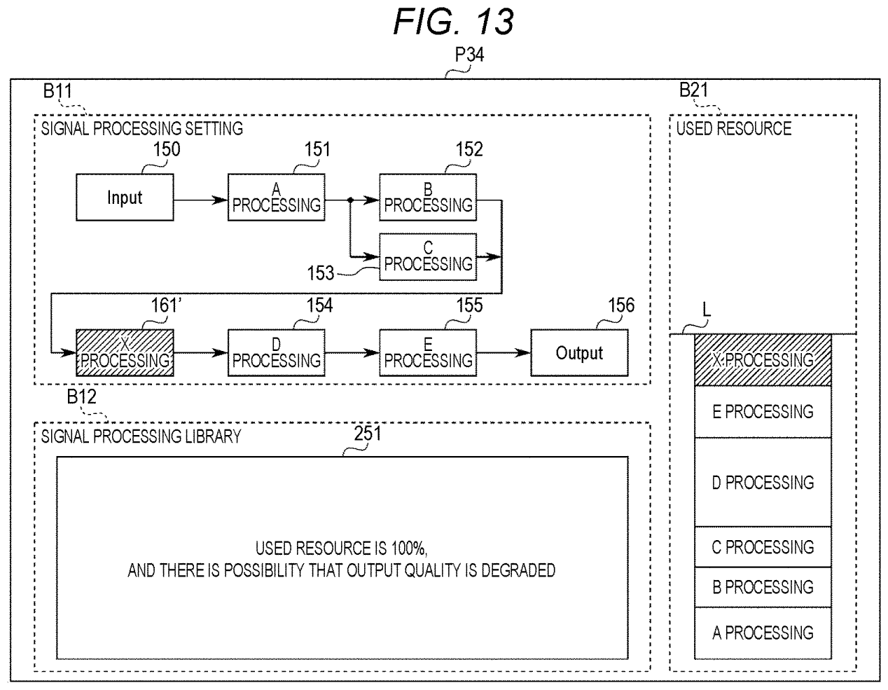

In a case where the set signal processing is executed, the quality confirmation processing unit 122″ may grasp the usage status of the resource related to the signal processing and display a warning image as illustrated by a setting image P34 in FIG. 13, for example, when recognizing that the usage status of the resource becomes 100% and there is no remaining resource.

In the setting image P34 of FIG. 13, a warning field 251 written as “Used resource is 100%, and there is a possibility that output quality is degraded” is displayed in the signal processing library field B12.

The warning image as illustrated in FIG. 13 indicates that, in the signal processing of the signal processing unit 41 in which the signal processing block 161′ indicated as “X processing” is newly selected, the used resource is saturated, and there is a possibility that quality of the output image deteriorates.

<Signal Processing by Quality Confirmation Unit in FIG. 9>

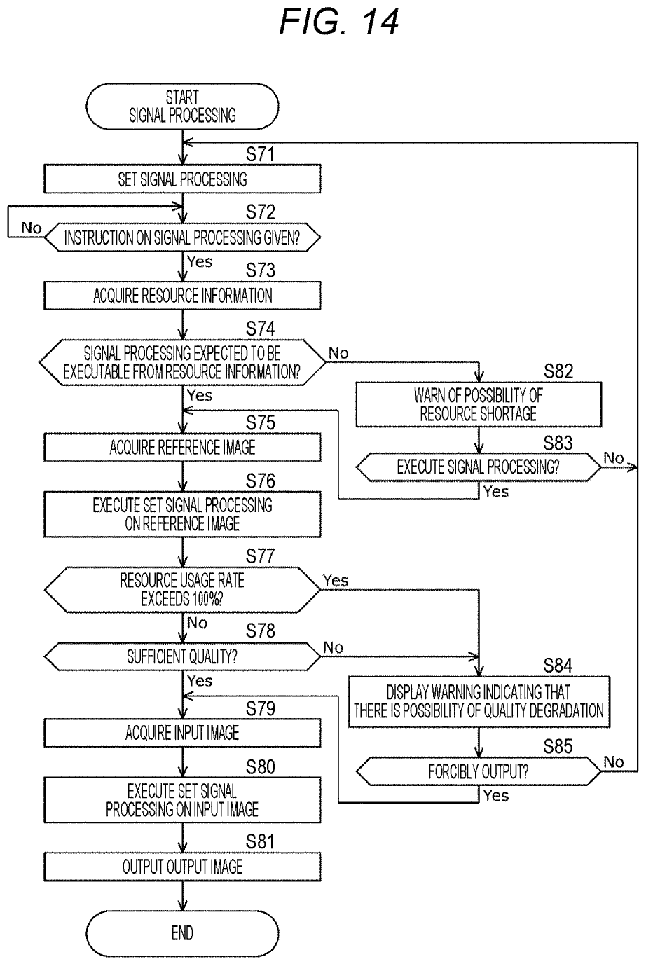

Next, signal processing by the quality confirmation unit 42 in FIG. 9 will be described with reference to a flowchart in FIG. 14.

In step S71, the quality confirmation processing unit 122″ displays a setting image as illustrated by a setting image P31 in FIG. 10 on the display unit 125, and receives an operation input of the operation unit 124, thereby setting contents of the signal processing of the signal processing unit 41.

In step S72, the quality confirmation processing unit 122″ determines whether or not the operation unit 124 has been operated and an instruction to cause the signal processing unit 41 to execute the set signal processing has been made, and repeats similar processing until the instruction to execute is made. Then, if the instruction on the signal processing by the signal processing unit 41 is given in step S72, the processing proceeds to step S73.

In step S73, the quality confirmation processing unit 122″ controls the resource management unit 222 to acquire information on resources of the signal processing unit 41.

In step S74, the quality confirmation processing unit 122″ estimates a resource to be used on the basis of the signal processing set in the signal processing unit 41, and compares the estimated resource with the information on the resources acquired from the resource management unit 222 to determine whether or not the resource is likely to be executable.

In a case where it is determined in step S74 that the set signal processing is expected to be executable for the resource, the processing proceeds to step S75.

In step S75, the quality confirmation processing unit 122″ controls the input unit 121 to acquire a reference image generated by the reference image generation unit 201.

In step S76, the quality confirmation processing unit 122″ controls the input unit 121 to supply the acquired reference image to the signal processing unit 41 to execute the set signal processing, and to supply the input image to itself. At this time, the signal processing unit 41 performs the set signal processing on the reference image to output an output image for the reference image as a signal processing result to the output unit 123.

In step S77, the quality confirmation processing unit 122″ acquires information on a resource usage rate supplied from the resource management unit 222, and determines whether or not the resource usage rate exceeds 100%.

More specifically, the resource management unit 222 controls the used resource measurement unit 221 to measure a current used resource in the signal processing unit 41, and supplies a measurement result to the quality confirmation processing unit 122″. In response to this, the quality confirmation processing unit 122″ determines whether or not the resource usage rate exceeds 100% on the basis of the measurement result of the current used resource supplied from the resource management unit 222.

In a case where it is determined in step S77 that the resource usage rate does not exceed 100%, the processing proceeds to step S78.

In step S78, the quality confirmation processing unit 122″ acquires the output image for the reference image that is the signal processing result of the signal processing unit 41 from the output unit 123, scores a difference between the reference image and the output image for the reference image, and determines whether or not an image quality change of the output image with respect to the input image is sufficiently small and the output image is an image of sufficient quality that satisfies criteria required for implementing medical practice from comparison between the obtained score and a predetermined threshold (first threshold).

In a case where it is determined in step S78 that the output image is an image of sufficient quality, the processing proceeds to step 79.

In step S79, the quality confirmation processing unit 122″ controls the input unit 121 to receive and acquire an input image from a designated input device.

In step S80, the quality confirmation processing unit 122″ controls the input unit 121 to supply the acquired input image to the signal processing unit 41 to execute the set signal processing, and to supply the input image to itself. At this time, the signal processing unit 41 performs the set signal processing on the input image to output an output image as a signal processing result to the output unit 123.

In step S81, the quality confirmation processing unit 122″ controls the output unit 123 to output and display the output image on the designated monitor device 51.

On the other hand, in a case where it is determined in step S74 that the signal processing set for the resource is not expected to be executable, the processing proceeds to step S82.

In step S82, the quality confirmation processing unit 122″ causes the display unit 125 to display a warning image indicating that there is a possibility that the signal processing cannot be executed due to an insufficient resource, for example, as described with reference to FIG. 11.

In step S83, the quality confirmation processing unit 122″ determines whether or not to execute the signal processing even if there is a possibility that the signal processing cannot be executed due to the insufficient resource.

Whether or not to execute the signal processing even if there is a possibility that the signal processing cannot be executed due to the insufficient resource may be inquired of a user every time or may be set in advance.

In a case where the signal processing is not executed in step S83, the processing returns to step S71, and processing contents of the signal processing of the signal processing unit 41 are set again.

On the other hand, in a case where the signal processing is executed in step S83, the processing proceeds to step S76, and the signal processing using the reference image is performed.

Furthermore, in a case where it is determined in step S77 that the resource usage rate exceeds 100%, or in a case where it is determined in step S78 that the output image is an image of sufficient quality, the processing proceeds to step 84.

In step S84, the quality confirmation processing unit 122″ controls the display unit 125 to display a warning image indicating that there is a possibility that the output image is not image quality of sufficient quality accompanied by quality degradation.

More specifically, in a case where it is determined in step S77 that the resource usage rate exceeds 100%, the quality confirmation processing unit 122″ displays, for example, a warning image including the warning field 231 in FIG. 13 in step S84. In a case where it is determined in step S78 that the output image is not an image of sufficient quality, the quality confirmation processing unit 122″ displays, for example, a warning image including the warning field 191 in FIG. 4 in step S84.

In step S85, the quality confirmation processing unit 122″ determines whether or not to forcibly execute the signal processing and output the output image even in a state where an image of sufficient quality cannot be obtained.

In a case where it is determined in step S85 that the signal processing is forcibly executed to output the image, the processing proceeds to step S79, and the output image is displayed on the designated monitor device 51 together with the warning image presented in the processing of step S84.

Furthermore, in a case where it is determined in step S85 that the signal processing is not forcibly executed to output the image, the processing returns to step S71, and the subsequent processing is repeated. That is, in this case, setting of the contents of the signal processing of the signal processing unit 41 is repeatedly changed until there is no resource shortage and it is considered that the output image satisfies sufficient quality or it is considered that the signal processing may be forcibly executed and the output image may be displayed on the assumption that the output image is displayed together with the warning image.

By the above processing, it is possible to freely set desired signal processing, and it is possible to present an output image of sufficient quality by determining whether or not the image quality change of the output image with respect to the input image is large by the set signal processing.

Since it is possible to confirm whether or not an image having sufficient quality is output as an output image by the set signal processing on the basis of the reference image in advance, it is possible to confirm quality of the output image with higher accuracy.

When the signal processing is set, it is possible to recognize the presence or absence of a possibility that the signal processing cannot be appropriately executed due to a resource shortage. Thus, it is possible to prevent the quality from being unintentionally deteriorated, and it is possible to immediately encourage the change of the processing contents of the signal processing before executing the signal processing using the reference image.

The warning image is presented not only whether or not an image of sufficient quality is output by executing the signal processing using the reference image, but also when the usage status of the resource at the time of actual signal processing is confirmed and the resource usage rate exceeds 100%. Therefore, it is possible to prevent unintentional deterioration in quality before executing the signal processing on the actual input image.

Since the resource is presented according to the order of importance of each signal processing module, it is possible to indicate an index indicating which signal processing module should be preferentially left in a case where an upper limit of the total resource is exceeded.

Therefore, for example, it is possible to perform setting such that the signal processing modules are rearranged centering on the signal processing module with high priority, and it is possible to easily realize resetting of signal processing such that safety is not affected.

Furthermore, for example, it is possible to prevent degradation of the quality of the output image, such as degradation of the display quality due to insufficient operation resources such as a CPU and a GPU and an increase in processing time less than or equal to a predetermined frame rate and latency.

Moreover, for example, it is possible to prevent that it takes more time for data access than usual since a memory capacity is insufficient and data is moved to a swap area such as an HDD and the like, a predetermined frame rate and latency cannot be achieved, and quality of an output image is deteriorated.

Note that, in the above processing, an example has been described in which the quality determination based on the usage rate of the resource when the signal processing is performed on the reference image and the quality determination based on the comparison between the reference image and the output image obtained by performing the signal processing on the reference image are performed after the expected quality from the resource is confirmed before the signal processing is performed.

However, the input image from the input device may be directly used without using the reference image for the quality determination.

That is, the quality determination based on the resource usage rate when the signal processing is performed on the input image from the input device and the quality determination based on the comparison between the input image and the output image obtained by performing the signal processing on the input image may be performed.

### 5. First Modification of Third Embodiment

<First Modification of Signal Processing by Quality Confirmation Unit in FIG. 9>

In the above description, an example has been described in which a warning image is presented in a case where there is a possibility that signal processing cannot be performed due to an insufficient resource before the signal processing using a reference image is executed and in a case where a resource is insufficient due to actual signal processing.

However, as a result of actually executing the signal processing using the reference image, in a case where there is a possibility of hindering a resource related to the signal processing with high importance (priority), a setting failure of the signal processing may be presented to urge resetting.

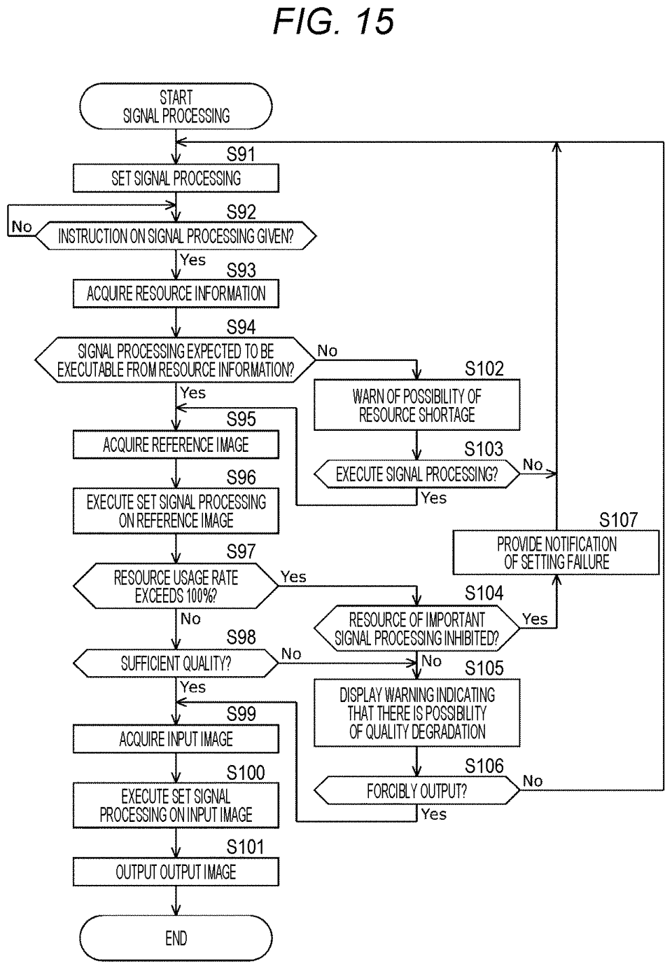

Here, with reference to a flowchart of FIG. 15, a first modification of the signal processing by the quality confirmation unit 42 of FIG. 9 will be described in which, in a case where there is a possibility of hindering a resource related to signal processing with high importance (priority) as a result of actually executing signal processing using a reference image, a setting failure of the signal processing is presented and resetting is urged.

Note that processing of steps S91 to S103, S105, and S106 in the flowchart of FIG. 15 is similar to the processing of steps S71 to S85 in the flowchart of FIG. 14, and thus description thereof is omitted.

That is, in the processing of steps S91 to S97, after the signal processing is set and the presence or absence of resource shortage is confirmed, the signal processing using the reference image is performed, and in a case where it is determined that a resource usage rate exceeds 100%, the processing proceeds to step S104.

In step S104, the quality confirmation processing unit 122″ acquires information on resources supplied from the resource management unit 222, and determines whether or not to inhibit the resource of the signal processing with high importance (priority).

More specifically, the resource management unit 222 controls the used resource measurement unit 221 to measure a current used resource in the signal processing unit 41 and supplies a measurement result to the quality confirmation processing unit 122″, and in response to this, the quality confirmation processing unit 122″ acquires the information on the resources supplied from the resource management unit 222.

The quality confirmation processing unit 122″ determines whether or not the resource of the signal processing having high importance (priority) is hindered on the basis of whether or not a resource of a signal processing module having higher importance (priority) than a predetermined level is secured among importance (priority) of individual signal processing modules constituting the set signal processing contents of the signal processing unit 41 supplied from the resource management unit 222.

The importance (priority) may be set in advance, or a user may set the importance (priority) when setting the signal processing of the signal processing unit 41.

In a case where it is determined in step S104 that the resource of the signal processing with high importance (priority) is inhibited, the processing proceeds to step S107.

In step S107, the quality confirmation processing unit 122″ controls the display unit 125 to display an image providing notification that the setting contents of the signal processing for the signal processing unit 41 cannot be set.

With this processing, in a case where the resource usage rate exceeds 100% and the resource of the signal processing with high importance (priority) is inhibited, there is a problem in quality of the signal processing of the signal processing unit 41 as a whole, and the signal processing with high importance cannot be appropriately realized. Therefore, it is possible to recognize that the setting itself of the processing contents of the signal processing of the signal processing unit 41 is impossible.

In other words, when the signal processing is performed in the signal processing unit 41 with the set processing contents, even if the resource exceeds 100%, unless the resource of the signal processing with high importance (priority) is inhibited, an output image of sufficient quality cannot be obtained, but the signal processing with high importance (priority) is appropriately performed. Therefore, it can be considered that there is no problem in the setting of the signal processing.

### 6. Second Modification of Third Embodiment

<Second Modification of Signal Processing by Quality Confirmation Unit in FIG. 9>

In the above description, an example has been described in which, in a case where a resource usage rate exceeds 100% and there is a possibility of hindering a resource related to signal processing with high importance (priority), a setting failure of signal processing of the signal processing unit 41 is presented, and resetting is urged.

However, in a case where a resource usage rate exceeds 100% or in a case where an image of sufficient quality cannot be output, quality of processing of a signal processing module with low importance among set signal processing is lowered or the processing is stopped, so that a resource may be secured and signal processing may be appropriately executed.

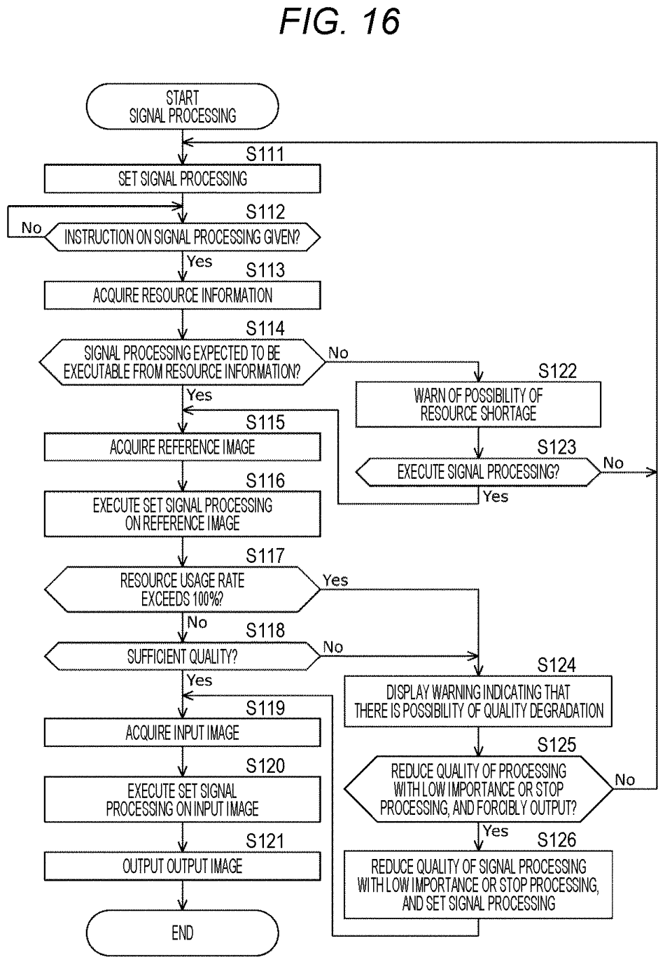

Here, with reference to a flowchart of FIG. 16, a second modification of the signal processing by the quality confirmation unit 42 of FIG. 9 will be described, in which, in a case where a resource usage rate exceeds 100% or in a case where an image of sufficient quality cannot be output, quality of processing of a signal processing module with low importance (priority) among set signal processing is lowered or the processing is stopped, thereby securing a resource and appropriately executing signal processing.

Note that processing of steps S111 to S124 in the flowchart of FIG. 16 is similar to the processing of steps S71 to S84 in the flowchart of FIG. 14, and thus description thereof is omitted.

That is, in the processing of steps S111 to S123, after the signal processing is set and the presence or absence of resource shortage is confirmed, the signal processing using a reference image is performed. It is determined that the resource usage rate exceeds 100%, or it is determined that the output image is not an image of sufficient quality. In step S124, when it is indicated that there is a possibility that the output image is not image quality of sufficient quality accompanied by quality degradation, the processing proceeds to step S125.

In step S125, for a signal processing module with low importance (priority) among currently set signal processing of the signal processing unit 41, the quality confirmation processing unit 122″ determines whether or not to execute the signal processing and output the output image by reducing a processing load to reduce quality or stopping the processing.

For the signal processing module with low importance (priority) among the currently set signal processing of the signal processing unit 41, whether or not to execute the signal processing and output the output image by reducing the processing load or stopping the processing may be inquired of a user every time or may be set in advance.

For the signal processing module with low importance (priority) among the currently set signal processing of the signal processing unit 41, in a case where the quality confirmation processing unit 122″ determines in step S125 that the signal processing is not executed and the output image is not output by reducing the processing load to reduce the quality or stopping the processing, the processing returns to step S111, and setting of new signal processing is promoted.

On the other hand, for the signal processing module with low importance (priority) among the currently set signal processing of the signal processing unit 41, in a case where the quality confirmation processing unit 122″ determines in step S125 that the signal processing is executed and the output image is output by reducing the processing load or stopping the processing, the processing proceeds to step S126.

In step S126, for the signal processing module with low importance (priority) among the currently set signal processing of the signal processing unit 41, the quality confirmation processing unit 122″ sets to change processing contents of the signal processing of the signal processing unit 41 so as to reduce the processing load or to stop the processing. The processing proceeds to step S119.

With this processing, for the signal processing module with low importance (priority) in the currently set signal processing of the signal processing unit 41, it is possible to realize the signal processing and display the output image on the desired monitor device 51 after suppressing a failure of the signal processing by the signal processing unit 41 by reducing the processing load or stopping the processing.

### 7. Example Executed by Software

Meanwhile, the series of processing described above can be executed by hardware or software. In a case where the series of processing is executed by the software, a program constituting the software is installed from a recording medium to a computer incorporated in dedicated hardware or, for example, a general-purpose computer or the like capable of executing various functions by installing various programs.

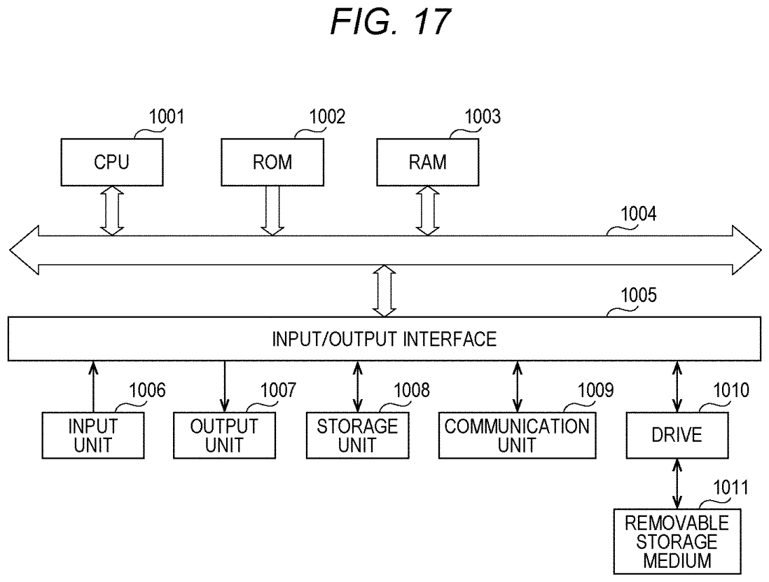

FIG. 17 is a diagram illustrating a configuration example of a general-purpose computer. This personal computer includes a central processing unit (CPU) 1001. An input/output interface 1005 is connected to the CPU 1001 via a bus 1004. A read only memory (ROM) 1002 and a random access memory (RAM) 1003 are connected to the bus 1004.

An input unit 1006 including an input device such as a keyboard and a mouse with which a user inputs an operation command, an output unit 1007 that outputs a processing operation screen and an image of a processing result to a display device, a storage unit 1008 including a hard disk drive or the like that stores a program and various data, and a communication unit 1009 including a local area network (LAN) adapter or the like and executing communication processing via a network represented by the Internet are connected to the input/output interface 1005. Furthermore, a drive 1010 that reads and writes data from and to a removable storage medium 1011 such as a magnetic disk (including a flexible disk), an optical disk (including a compact disc-read only memory (CD-ROM) and a digital versatile disc (DVD)), a magneto-optical disk (including a mini disc (MD)), or a semiconductor memory is connected.

The CPU 1001 executes various processing according to a program stored in the ROM 1002 or a program read from a removable storage medium 1011 such as a magnetic disk, an optical disk, a magneto-optical disk, a semiconductor memory, and the like installed in the storage unit 1008, and loaded from the storage unit 1008 to the RAM 1003. Furthermore, the RAM 1003 appropriately stores data and the like necessary for the CPU 1001 to execute various processing.

In the computer configured as described above, for example, the CPU 1001 loads a program stored in the storage unit 1008 into the RAM 1003 via the input/output interface 1005 and the bus 1004 and executes the program, whereby the above-described series of processing is performed.

The program executed by the computer (CPU 1001) can be provided by recording on the removable storage medium 1011 as a package medium and the like, for example. Furthermore, the program can be provided via a wired or wireless transmission medium such as a local area network, the Internet, or digital satellite broadcasting.

In the computer, the program can be installed in the storage unit 1008 via the input/output interface 1005 by attaching the removable storage medium 1011 to the drive 1010. Furthermore, the program can be received by the communication unit 1009 via a wired or wireless transmission medium and installed in the storage unit 1008. In addition, the program can be installed in the ROM 1002 or the storage unit 1008 in advance.

Note that the program executed by the computer may be a program in which processing is performed in time series in the order described in the present specification, or may be a program in which processing is performed in parallel or at necessary timing such as when a call is made, and the like.

Note that the CPU 1001 in FIG. 17 implements the functions of the quality confirmation processing unit 122 in FIG. 2, the quality confirmation processing unit 122′ in FIG. 6, and the quality confirmation processing unit 122″ in FIG. 9.

Furthermore, in the present specification, the system means a set of a plurality of components (devices, modules (parts), and the like), and it does not matter whether or not all the components are in the same housing. Therefore, a plurality of devices housed in separate housings and connected via a network, and one device housing a plurality of modules in one housing are both systems.

Note that the embodiment of the present disclosure is not limited to the above-described embodiments, and various modifications can be made without departing from the gist of the present disclosure.

For example, the present disclosure can be configured as cloud computing in which one function is shared and jointly processed by a plurality of devices via a network.

Furthermore, each step described in the above-described flowcharts can be executed by one device or shared and executed by a plurality of devices.

Moreover, in a case where one step includes a plurality of processing, the plurality of processing included in the one step can be executed by one device or shared and executed by a plurality of devices.

Note that the present disclosure can also have the following configurations.
<1> A medical information processing system including:
a quality confirmation unit configured to confirm quality of output medical information which is medical information obtained by performing predetermined signal processing on input medical information which is medical information that has been input.
<2> The medical information processing system according to <1>, in which
the quality confirmation unit confirms whether or not the quality of the output medical information is sufficient on the basis of a difference between the input medical information and the output medical information.
<3> The medical information processing system according to <2>, in which
in a case where the difference between the input medical information and the output medical information is larger than a first threshold, the quality confirmation unit considers that the quality of the output medical information is not sufficient, presents a warning indicating that the quality of the output medical information is not sufficient, and stops output of the output medical information.
<4> The medical information processing system according to <3>, in which
in a case where the difference between the input medical information and the output medical information is larger than the first threshold and smaller than a second threshold larger than the first threshold, the quality of the output medical information is not sufficient, but the quality confirmation unit presents a warning indicating that the quality of the output medical information is not sufficient, and outputs the output medical information.
<5> The medical information processing system according to any one of <1> to <4>, in which
an output destination of the output medical information is set in the predetermined signal processing, and
the quality confirmation unit confirms whether or not the output medical information has appropriate quality for the output destination.
<6> The medical information processing system according to <1>, in which
the quality confirmation unit confirms the quality of the output medical information on the basis of whether or not the predetermined signal processing is processing conforming to the Pharmaceutical Affairs Law.
<7> The medical information processing system according to any one of <1> to <6>, in which
the quality confirmation unit confirms the quality of the output medical information by comparing input reference information which is reference information that has been input and output reference information which is reference information obtained by performing the predetermined signal processing on the input reference information.
<8> The medical information processing system according to <7>, in which
the quality confirmation unit confirms whether or not the quality of the output medical information is sufficient on the basis of a difference between the input reference information and the output reference information.
<9> The medical information processing system according to <7>, in which
when confirming that the quality of the output medical information is sufficient by comparing the input reference information and the output reference information, the quality confirmation unit generates and outputs the output medical information so that the predetermined signal processing is performed on the input medical information.
<10> The medical information processing system according to <7>, in which
the quality confirmation unit receives a setting operation of setting processing contents of the predetermined signal processing, sets the processing contents of the predetermined signal processing by selecting and connecting processing modules configuring the predetermined signal processing, and presents that the processing contents of the predetermined signal processing cannot be set when it is confirmed that there is a fatal problem in the quality of the output medical information by comparing the input reference information and the output reference information.
<11> The medical information processing system according to any one of <1> to <10>, in which
the quality confirmation unit confirms the quality of the output medical information on the basis of a resource related to the predetermined signal processing.
<12> The medical information processing system according to <11>, in which
the quality confirmation unit confirms whether or not the quality of the output medical information is sufficient on the basis of whether or not the resource related to the predetermined signal processing is insufficient.
<13> The medical information processing system according to <11>, in which
the quality confirmation unit receives a setting operation of setting processing contents of the predetermined signal processing, sets the processing contents of the predetermined signal processing by selecting and connecting processing modules that execute individual signal processing, and confirms the quality of the output medical information by comparing input reference information which is reference information that has been input and output reference information which is reference information obtained by performing the predetermined signal processing on the input reference information, in a case where the resource related to the predetermined signal processing is not insufficient on the basis of the set processing contents.
<14> The medical information processing system according to <11>, in which
the quality confirmation unit receives a setting operation of setting processing contents of the predetermined signal processing, sets the processing contents of the predetermined signal processing by selecting and connecting processing modules configuring the predetermined signal processing, and presents that the predetermined signal processing cannot be set in a case where the resource related to the predetermined processing module is hindered on the basis of the set processing contents.
<15> The medical information processing system according to <14>, in which
in a case where the resource related to the processing module having importance higher than predetermined importance among the processing modules is hindered, the quality confirmation unit presents that the predetermined signal processing cannot be set.
<16> The medical information processing system according to <11>, in which
the quality confirmation unit receives a setting operation of setting the processing contents of the predetermined signal processing, sets the processing contents of the predetermined signal processing by selecting and connecting processing modules configuring the predetermined signal processing, and reduces or stops a processing load of the processing module having importance lower than predetermined importance among the processing modules, in a case where the resource related to the predetermined signal processing is insufficient on the basis of the set processing contents.
<17> The medical information processing system according to <11>, in which
the resource includes at least one of operation resources such as a central processing unit (CPU), a graphics processing unit (GPU), a digital signal processor (DSP), and an encoder/decoder used for executing the predetermined signal processing, a memory capacity/band resource, an internal transmission bus band resource, an amount of the resource serving as a reference of communication band resource, or a current usage rate of the resource.
<18> The medical information processing system according to any one of <1> to <17>, in which
the input medical information and the output medical information are medical images, and
the quality confirmation unit confirms quality of the medical images based on at least one of an increase in latency, a decrease or improvement (stretching) in resolution, a change in frame rate, a change in display luminance, image degradation, image addition, or overlapping of a plurality of images.
<19> A medical information processing method including:
confirming quality of output medical information which is medical information obtained by performing predetermined signal processing on input medical information which is medical information that has been input.
<20> A program causing a computer to function as:
a quality confirmation unit configured to confirm quality of output medical information which is medical information obtained by performing predetermined signal processing on input medical information which is medical information that has been input.

### REFERENCE SIGNS LIST

11 Medical information processing system
31 Endoscope device
32 Vital monitor
33 CT device
34 Medical record management device
41 Signal processing unit
42 Quality confirmation unit
51, 51-1 to 51-4 Monitor device
101 A processing module
102 B processing module
103 C processing module
104 D processing module
105 E processing module
121 Input unit
122, 122′, 122″ Quality confirmation processing unit
123 Output unit
124 Operation unit
125 Display unit
201 Reference image generation unit
221 Used resource measurement unit
222 Resource management unit

1. A medical information processing system comprising:
a quality confirmation unit configured to confirm quality of output medical information which is medical information obtained by performing predetermined signal processing on input medical information which is medical information that has been input.

2. The medical information processing system according to claim 1, wherein
the quality confirmation unit confirms whether or not the quality of the output medical information is sufficient on a basis of a difference between the input medical information and the output medical information.

3. The medical information processing system according to claim 2, wherein
in a case where the difference between the input medical information and the output medical information is larger than a first threshold, the quality confirmation unit considers that the quality of the output medical information is not sufficient, presents a warning indicating that the quality of the output medical information is not sufficient, and stops output of the output medical information.

4. The medical information processing system according to claim 3, wherein
in a case where the difference between the input medical information and the output medical information is larger than the first threshold and smaller than a second threshold larger than the first threshold, the quality of the output medical information is not sufficient, but the quality confirmation unit presents a warning indicating that the quality of the output medical information is not sufficient, and outputs the output medical information.

5. The medical information processing system according to claim 1, wherein
an output destination of the output medical information is set in the predetermined signal processing, and
the quality confirmation unit confirms whether or not the output medical information has appropriate quality for the output destination.

6. The medical information processing system according to claim 1, wherein
the quality confirmation unit confirms the quality of the output medical information on a basis of whether or not the predetermined signal processing is processing conforming to the Pharmaceutical Affairs Law.

7. The medical information processing system according to claim 1, wherein
the quality confirmation unit confirms the quality of the output medical information by comparing input reference information which is reference information that has been input and output reference information which is reference information obtained by performing the predetermined signal processing on the input reference information.

8. The medical information processing system according to claim 7, wherein
the quality confirmation unit confirms whether or not the quality of the output medical information is sufficient on a basis of a difference between the input reference information and the output reference information.

9. The medical information processing system according to claim 7, wherein
when confirming that the quality of the output medical information is sufficient by comparing the input reference information and the output reference information, the quality confirmation unit generates and outputs the output medical information so that the predetermined signal processing is performed on the input medical information.

10. The medical information processing system according to claim 7, wherein
the quality confirmation unit receives a setting operation of setting processing contents of the predetermined signal processing, sets the processing contents of the predetermined signal processing by selecting and connecting processing modules configuring the predetermined signal processing, and presents that the processing contents of the predetermined signal processing cannot be set when it is confirmed that there is a fatal problem in the quality of the output medical information by comparing the input reference information and the output reference information.

11. The medical information processing system according to claim 1, wherein
the quality confirmation unit confirms the quality of the output medical information on a basis of a resource related to the predetermined signal processing.

12. The medical information processing system according to claim 11, wherein
the quality confirmation unit confirms whether or not the quality of the output medical information is sufficient on a basis of whether or not the resource related to the predetermined signal processing is insufficient.

13. The medical information processing system according to claim 11, wherein
the quality confirmation unit receives a setting operation of setting processing contents of the predetermined signal processing, sets the processing contents of the predetermined signal processing by selecting and connecting processing modules that execute individual signal processing, and confirms the quality of the output medical information by comparing input reference information which is reference information that has been input and output reference information which is reference information obtained by performing the predetermined signal processing on the input reference information, in a case where the resource related to the predetermined signal processing is not insufficient on a basis of the set processing contents.

14. The medical information processing system according to claim 11, wherein
the quality confirmation unit receives a setting operation of setting processing contents of the predetermined signal processing, sets the processing contents of the predetermined signal processing by selecting and connecting processing modules configuring the predetermined signal processing, and presents that the predetermined signal processing cannot be set in a case where the resource related to the predetermined processing module is hindered on a basis of the set processing contents.

15. The medical information processing system according to claim 14, wherein
in a case where the resource related to the processing module having importance higher than predetermined importance among the processing modules is hindered, the quality confirmation unit presents that the predetermined signal processing cannot be set.

16. The medical information processing system according to claim 11, wherein
the quality confirmation unit receives a setting operation of setting the processing contents of the predetermined signal processing, sets the processing contents of the predetermined signal processing by selecting and connecting processing modules configuring the predetermined signal processing, and reduces or stops a processing load of the processing module having importance lower than predetermined importance among the processing modules, in a case where the resource related to the predetermined signal processing is insufficient on a basis of the set processing contents.

17. The medical information processing system according to claim 11, wherein
the resource includes at least one of operation resources such as a central processing unit (CPU), a graphics processing unit (GPU), a digital signal processor (DSP), and an encoder/decoder used for executing the predetermined signal processing, a memory capacity/band resource, an internal transmission bus band resource, an amount of the resource serving as a reference of a communication band resource, or a current usage rate of the resource.

18. The medical information processing system according to claim 1, wherein
the input medical information and the output medical information are medical images, and
the quality confirmation unit confirms quality of the medical images based on at least one of an increase in latency, a decrease or improvement (stretching) in resolution, a change in frame rate, a change in display luminance, image degradation, image addition, or overlapping of a plurality of images.

19. A medical information processing method comprising:
confirming quality of output medical information which is medical information obtained by performing predetermined signal processing on input medical information which is medical information that has been input.

20. A program causing a computer to function as:
a quality confirmation unit configured to confirm quality of output medical information which is medical information obtained by performing predetermined signal processing on input medical information which is medical information that has been input.
